(19) United States
(12) Patent Application Publication
(10) Pub. No.: US 2006/0141569 A1
(43) Pub. Date: Jun. 29, 2006

(54) Compositions and methods for the assay of G-protein coupled receptors and their ligands

(76) Inventor: Matthew Hsu, Escondido, CA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/262,484

(22) Filed: Oct. 28, 2005

Related U.S. Application Data
(60) Provisional application No. 60/623,418, filed on Oct. 29, 2004.

(51) Int. Cl. C07K 14/705; C07H 21/04; C12P 21/06

(52) U.S. Cl. 435/69.1; 435/358; 435/320.1; 530/350; 536/23.5

(57) ABSTRACT

A cell comprising one or more endogenous promiscuous G-proteins and an exogenous nucleic acid sequence that encodes a G-protein coupled receptor (GPCR), which optionally comprises a calcium release activated calcium channel (CRAC channel) provides features and improvements that significantly improve membrane level expression of the GPCR, increase signal responses and duration of signal, and decrease background in GPCR-related assay applications.

subunits encoded by distinct genes. The α subunit is responsible for the binding of GDP and GTP. Binding of a ligand to a GPCR results in a transition of the a subunit from a GDP-bound form to a GTP-bound form and leads to the activation of the heterotrimer through dissociation of the α-GTP from the βγ dimer. Both α-GTP and the βγ dimer regulate the activities of a variety of effectors that transmit the signal to the cell interior through the production of second messenger molecules (e.g., calcium, cAMP, etc). There are at least 17 Gα genes, and members of G proteins can be grouped into four main classes termed Gα_{i/0}, Gα_{q}, Gαₛ and Gα₁₂. GPCRs come in many different flavors, and upon ligand binding they can couple to a variety of G proteins, thus leading to activation of many complex signaling pathways, which can complicate the readout for High Throughput Screening (HTS) of GPCRs.

Fluorometric Imaging Plate Reader (FLIPR®) and aequorin technologies provide rapid and sensitive read-out for many GPCR drug targets and have become the systems of choice for measuring the changes in intracellular calcium upon binding of ligands to GPCRs in a high throughput manner. Not all GPCRs, however, couple to Gα_{q} and thereby activate the PLCβ pathway leading to calcium mobilization.

The conventional method for coupling a non-Gα_{q} coupled receptor to the PLCβ/calcium pathway is to co-transfect either promiscuous G protein (Gα₁₅ and/or Gα₁₆) or Gα_{q} chimeras to promote FLIPR® or Aequorin readout. However, there are many limitations and drawbacks to this method. For example, it is difficult to control and normalize the amount of G proteins that are transfected, which can cause a significant shift of the ligand EC50 and a change in the structure-activity relationship (SAR). In addition, overexpression of recombinant Gα₁₅ and/or Gα₁₆ can cause constitutive activation of GPCRs and thereby generate high background, which can hinder analysis.

Accordingly, improved methods for identifying and characterizing GPCRs are needed.

### SUMMARY OF THE INVENTION

The present invention is based on the discovery that particular cells comprise one or more endogenous promiscuous G-proteins expressed at a high level, and that such cells can express at a high level one or more GPCRs that are encoded by an exogenous nucleic acid sequence. The invention is further based on the discovery of a novel mammalian expression vector, pHS, which is compatible with the cells of the invention and results in a significant increase in the level of GPCR expression on the cell surface and functional coupling of the expressed GPCR to the endogenous promiscuous G-protein and superior calcium mobilization.

In one embodiment, the invention is a cell comprising one or more endogenous promiscuous G-proteins and an exogenous nucleic acid sequence that encodes a (one or more) G-protein coupled receptor (GPCR), wherein the one or more G-proteins and the GPCR are expressed at high levels. In a particular embodiment, the cell further comprises an endogenous calcium release activated calcium (CRAC) channel.

In another embodiment, the invention is pHS vector, deposited at the American Type Culture Collection (ATCC) as Accession Number PTA-6986. In still another embodiment, the invention is a cell transfected with pHS vector.

The invention is also directed to a method of identifying an agent that increases activity of a G-protein coupled receptor (GPCR). The method comprises combining a cell that comprises one or more endogenous promiscuous G-proteins and an exogenous nucleic acid sequence that encodes a G-protein coupled receptor (GPCR), wherein the one or more G-proteins and the GPCR are expressed at high levels and a test agent, and detecting activity of the GPCR. In the method, an increase in activity of the GPCR, relative to a control, indicates that the test agent increases activity of the GPCR.

A method of identifying a ligand of a G-protein coupled receptor (GPCR) is also provided herein. In this method, a cell that comprises one or more endogenous promiscuous G-proteins and an exogenous nucleic acid sequence that encodes a G-protein coupled receptor (GPCR), wherein the one or more G-proteins and the GPCR are expressed at high levels, is combined with a test ligand. Activity of the GPCR is then detected. In the method, an increase in activity of the GPCR, relative to a control, indicates that the test ligand is a ligand of the GPCR. In a particular embodiment, the GPCR is an orphan GPCR.

The invention is also directed to a method of identifying an agent that modulates activity of a G-protein coupled receptor (GPCR). The method comprises combining a cell that comprises one or more endogenous promiscuous G-proteins and an exogenous nucleic acid sequence that encodes a G-protein coupled receptor (GPCR), wherein the one or more G-proteins and the GPCR are expressed at high levels, an agent that activates the GPCR, and a test agent. Activity of the GPCR is then detected. In the method, an alteration in activity of the GPCR, relative to a control, indicates that the test agent modulates activity of the GPCR.

The invention also provides a method of expressing a G-protein coupled receptor (GPCR) in a cell, comprising transfecting the cell with a nucleic acid sequence encoding the GPCR, wherein the cell comprises one or more endogenous promiscuous G-proteins. The cell (transfected cell) is maintained under conditions in which the one or more G-proteins and the GPCR are expressed at high levels.

Also encompassed by the invention is a method of measuring an alteration in intracellular calcium in a cell. The method comprises combining a cell comprising one or more endogenous promiscuous G-proteins and an exogenous nucleic acid sequence that encodes a G-protein coupled receptor (GPCR), wherein the one or more G-proteins and the GPCR are expressed at high levels and an agent that activates the GPCR. Intracellular calcium in the cell is then measured. The intracellular calcium that has been measured can be compared to a suitable control to show alteration of the intracellular calcium in the cell.

The invention is also directed to a method of coupling a G-protein coupled receptor (GPCR) to the PLCβ pathway. The method comprises combining a cell that comprises one or more endogenous promiscuous G-proteins and an exogenous nucleic acid sequence that encodes a G-protein coupled receptor (GPCR) and an agent that activates the GPCR, under conditions in which the GPCR is activated. In the method, the one or more G-proteins and the GPCR are expressed at high levels. The one or more G-proteins can be selected from the group consisting of a G protein of the Gα_{i/0} family, a G protein of the Gαₛ family and a G protein of the Gα₁₂ family.

### BRIEF DESCRIPTION OF THE DRAWINGS

FIG. 1 is a schematic showing conventional methods for coupling non-Gα_{q} coupled GPCRs to the PLCβ/Calcium pathway. As depicted, co-transfection of the promiscuous Gα₁₅ and/or Gα₁₆ G protein(s) or Gq chimeras activates PLCβ, which leads to IP₃ production and calcium mobilization, and allows for FLIPR® or aequorin readout.

FIGS. 2A and 2B are graphs depicting the limitations of co-transfecting Gα₁₆ and a GPCR to promote the Ca²⁺ response for non-Gq-coupled GPCRs. Specifically, varying amounts of Gα₁₆ (0.5, 1, 2, 3, 4 and 5 μg) as well as a vector control were co-transfected with the Gαᵢ coupled GPCR, C5aR, into Chinese Hamster Ovary (CHO) cells to promote the FLIPR® calcium response. FIG. 2A shows the % of maximal FLIPR® response plotted against the Log M [C5a]. FIG. 2B depicts the maximal FLIPR® response (as a %) plotted against the amount of Gα₁₆ that was transfected.

FIG. 3 is an ethidium-stained agarose gel showing the level of Gα₁₅ and/or Gα₁₆ RT-PCR product for particular cell lines (i.e., Chem-1 (RBL-2H3 cells; ATCC Accession No. CRL-2256), HL60 cells, THP-1 cells, Chem-2 (U937 cells; ATCC Accession No. CRL-1593.2), 70Z/3 cells, Jurkat cells, Chem-3 cells (BA/F3 cells; DSMZ Accession No. ACC300), Hela cells, HEK293 cells and CHO cells).

FIG. 4 is a schematic representation of the mammalian expression vector pHS. pHS, which is a pBluescript®-derived plasmid, contains a non-CMV promoter (Spleen Focus Forming Virus (SFFV) LTR) and an endoplasmic reticulum (ER) export signal and provides for high level expression of GPCRs.

FIG. 5A is a confocal microscopy fluorescent image depicting expression of a CXCR2-GFP fusion protein expressed using the pcDNA3 mammalian expression vector in CHO cells.

FIG. 5B is a confocal microscopy fluorescent image depicting expression of a CXCR2-GFP fusion protein expressed using the pHS expression vector in CHEM-1 cells.

FIG. 6A is a fluorescence plot (FACs analysis) showing expression of a CXCR2-GFP fusion protein expressed using the pcDNA3 mammalian expression vector in CHO cells.

FIG. 6B is a fluorescence plot (FACs analysis) showing expression of a CXCR2-GFP fusion protein expressed using the pHS expression vector in CHEM-1 cells.

FIG. 7A is a graph depicting CXCR2 membrane radioligand saturation binding for membranes of CHO cells transfected with CXCR2 encoded by the mammalian expression vector pcDNA3. Specifically, 5 μg of GPCR membrane preps were incubated with increasing concentrations (0-2 nM) of ¹²⁵I-labeled ligand Groα (PerkinElmer, Wellesley, Mass.) in the absence or presence of an excess amount of cold ligand Groα. As depicted in FIG. 7A, the K_{D} was 0.1484 and the Bmax was 2756 fmol/mg.

FIG. 7B is a graph depicting CXCR2 membrane radioligand saturation binding for membranes of CHEM-1 cells transfected with CXCR2 encoded by the expression vector pHS. Specifically, 5 μg of GPCR membrane preparation was incubated with increasing concentrations (0-2 nM) of ¹²⁵I-labeled ligand Groα (PerkinElmer, Wellesley, Mass.) in the absence or presence of an excess amount of cold ligand Groα. As depicted in FIG. 7B, the K_{D} was 0.2787 and the Bmax was 51122 fmol/mg.

FIG. 8A is a graph depicting a radioligand (¹²⁵I-labeled Groα) competition binding assay for membranes of CHO cells transfected with CXCR2 encoded by the mammalian expression vector pcDNA3. Specifically, 5 μg of GPCR membrane preparation was incubated with 0.5 nM (˜Ki value) of ¹²⁵I-labeled ligand Groα (PerkinElmer, Wellesley, Mass.) and increasing concentrations (1×10⁻¹² to 1×10⁻⁶) of cold competitor (Groα or IL-8) in a 96-well plate. After 2 hours of incubation at room temperature, membranes were harvested to a 96-well GF/C filter plate, dried and counted using a Microbeta counter. As depicted, the conventional pcDNA3/CHO cell expression system generated membranes that exhibited a two site binding curve.

FIG. 8B is a graph depicting a radioligand (¹²⁵I-labeled Groα) competition binding assay for membranes of CHEM-1 cells transfected with CXCR2 encoded by the mammalian expression vector pcDNA3. Specifically, 5 μg of GPCR membrane preparation was incubated with 0.5 nM (˜Ki value) of ¹²⁵I-labeled ligand Groα (PerkinElmer, Wellesley, Mass.) and increasing concentrations (1×10⁻¹² to 1×10⁻⁶) of cold competitor (Groα or IL-8) in a 96-well plate. After 2 hours of incubation at room temperature, membranes were harvested to a 96-well GF/C filter plate, dried and counted using a Microbeta counter. As depicted, the pHS/CHEM-1 cell expression system generated membranes that exhibited a one site binding curve.

FIG. 9A is a graph depicting ligand (Groα)-induced calcium mobilization through endogenous CXCR2 in HL60 neutrophils in the absence of pertussis toxin (−PTX). Specifically, cells were loaded with the ratiometric fluorescent calcium dye Indo-1 (Molecular Probes/InVitrogen, Carlsbad, Calif.) and incubated for 30 min at 37° C. in the dark with Pluronic F-127 and Probenecid in HBSS buffer (with Ca²⁺ and Mg²⁺). The cells were then washed twice with HBSS and assayed for calcium mobilization in a single cuvette on a fluorometer upon ligand addition. TX-100=a detergent that releases the entire calcium store in the cell.

FIG. 9B is a graph depicting ligand (Groα)-induced calcium mobilization through endogenous CXCR2 in HL60 neutrophils in the presence of pertussis toxin (+PTX; 100 ng/ml). Specifically, cells were loaded with the ratiometric fluorescent calcium dye Indo-1 (Molecular Probes/InVitrogen, Carlsbad, Calif.) and incubated for 30 min at 37° C. in the dark with Pluronic F-127 and Probenecid in HBSS buffer (with Ca²⁺ and Mg²⁺). The cells were then washed twice with HBSS and assayed for calcium mobilization in a single cuvette on a fluorometer upon ligand addition. TX-100=a detergent that releases the entire calcium store in the cell.

FIG. 9C is a graph depicting ligand (Groα)-induced calcium mobilization through endogenous CXCR2 in HL60 neutrophils in the presence of a dominant negative Gα₁₅ (DNGα₁₅; 2 μg/well). Specifically, cells were transiently transfected with 2 μg/well of dominant negative Gα₁₅ (DNGα₁₅) and pre-incubated with PTX before ligand addition. TX-100=a detergent that releases the entire calcium store in the cell.

FIG. 9D is a graph depicting ligand (Groα)-induced calcium mobilization through CXCR2-transfected CHO cells in the absence of pertussis toxin (−PTX). Specifically, cells were loaded with the ratiometric fluorescent calcium dye Indo-1 (Molecular Probes/InVitrogen, Carlsbad, Calif.) and incubated for 30 min at 37° C. in the dark with Pluronic F-127 and Probenecid in HBSS buffer (with Ca²⁺ and Mg²⁺). The cells were then washed twice with HBSS and assayed for calcium mobilization in a single cuvette on a fluorometer upon ligand addition.

FIG. 9E is a graph depicting ligand (Groα)-induced calcium mobilization through CXCR2-transfected CHO cells in the presence of pertussis toxin (+PTX; 100 ng/ml). Specifically, cells were loaded with the ratiometric fluorescent calcium dye Indo-1 (Molecular Probes/InVitrogen, Carlsbad, Calif.) and incubated for 30 min at 37° C. in the dark with Pluronic F-127 and Probenecid in HBSS buffer (with Ca²⁺ and Mg²⁺). The cells were then washed twice with HBSS and assayed for calcium mobilization in a single cuvette on a fluorometer upon ligand addition.

FIG. 9F is a graph depicting ligand (Groα)-induced calcium mobilization through CXCR2-transfected CHO cells in the presence of a dominant negative Gα₁₅ (DNGα₁₅; 2 μg/well). Specifically, cells were transiently transfected with 2 μg/well of dominant negative Gα₁₅ (DNGα₁₅) and pre-incubated with PTX before ligand addition.

FIG. 9G is a graph depicting ligand (Groα)-induced calcium mobilization through CXCR2-transfected CHEM-1 cells in the absence of pertussis toxin (−PTX). Specifically, cells were loaded with the ratiometric fluorescent calcium dye Indo-1 (Molecular Probes/InVitrogen, Carlsbad, Calif.) and incubated for 30 min at 37° C. in the dark with Pluronic F-127 and Probenecid in HBSS buffer (with Ca²⁺ and Mg²⁺). The cells were then washed twice with HBSS and assayed for calcium mobilization in a single cuvette on a fluorometer upon ligand addition.

FIG. 9H is a graph depicting ligand (Groα)-induced calcium mobilization through CXCR2-transfected CHEM-1 cells in the presence of pertussis toxin (+PTX; 100 ng/ml). Specifically, cells were loaded with the ratiometric fluorescent calcium dye Indo-1 (Molecular Probes/InVitrogen, Carlsbad, Calif.) and incubated for 30 min at 37° C. in the dark with Pluronic F-127 and Probenecid in HBSS buffer (with Ca²⁺ and Mg²⁺). The cells were then washed twice with HBSS and assayed for calcium mobilization in a single cuvette on a fluorometer upon ligand addition.

FIG. 9I is a graph depicting ligand (Groα)-induced calcium mobilization through CXCR2-transfected CHEM-1 cells in the presence of a dominant negative Gα₁₅ (DNGα₁₅; 2 μg/well). Specifically, cells were transiently transfected with 2 μg/well of dominant negative Gα₁₅ (DNGα₁₅) and pre-incubated with PTX before ligand addition.

FIG. 10A is a graph depicting a FLIPR® multiple well average overlay ligand (SST-14) dose response for the Gαᵢ-coupled GPCR, SSTR2, which was transfected into CHEM-1 cells using the pHS vector.

FIG. 10B is a graph depicting a FLIPR® ligand (SST-14) dose response for the Gi-coupled GPCR, SSTR2, which was transfected into CHEM-1 cells using the pHS vector.

FIG. 10C is a graph comparing the C5aR FLIPR® dose response and ligand EC50 in C5aR-transfected CHEM-1 cells using the pHS vector (labeled as “C5aR/Chem-1”) and CHO cells co-transfected with Gα₁₅ and C5aR using the pcDNA3 vector (labeled as “C5aR+Gα15/CHO”). As depicted in FIG. 10C, CHEM-1 cells give an EC50 value that is consistent with the Ki binding value.

FIG. 10D is a graph depicting the agonist-induced CB1 receptor FLIPR® dose response for CB1-transfected CHEM-1 cells using the pHS vector. As depicted, the dose response for a full agonist (WIN55212) and partial agonists (CP55940) are shown and give EC50 values that are consistent with the Ki binding value.

FIG. 11 is a schematic depicting amplification of the intracellular Ca²⁺ signal by activation of store-operated Calcium Release Activated Calcium (CRAC) channels endogenously expressed in CHEM-2 and CHEM-3 Cells. APB=an IP₃R specific inhibitor; GSK96365=CRAC channel inhibitor.

FIG. 12A is a graph depicting an overlay of a FLIPR® minigraph depicting a ligand (MIP-3β)-induced CCR7-mediated FLIPR® dose response for CCR7-transfected CHEM-2 cells.

FIG. 12B is a graph depicting a ligand (MIP-3β)-induced CCR7-mediated FLIPR® dose response for various cell lines with or without promiscuous G protein coupling and CRAC channels. CCR7/Chem-2-Gα16/CRAC=CCR7-transfected CHEM-2 cells expressing endogenous Gα₁₆ and CRAC channel; Priess B cells-Gα16 coupling=endogenous CCR7 expressed in Priess B cells expressing Gα₁₆ but no CRAC channel; PHA T cell-Gi/βγ coupling=endogenous CCR7 expressed in T cells without any Gα₁₆ or CRAC channel but mediated through Gβγ subunits dissociated from Gαᵢ; CCR7/HEK293-Gi/βγ coupling=CCR7 transfected HEK293 cells without any Gα₁₆ or CRAC channel, but mediated through Gβγ subunits dissociated from Gαᵢ.

FIG. 12C is a graph depicting inhibition of ligand (MIP-3β)-induced CCR7-mediated calcium flux by a small molecule antagonist (400009). 400009=a CCR7 small molecule antagonist; activated T cells=T cells activated by phytohemagglutin (PHA).

FIG. 12D is a graph depicting inhibition of ligand (MIP-3β)-induced CCR7-mediated chemotaxis by a small molecule antagonist (400009). 400009=a CCR7 small molecule antagonist; activated T cells=T cells activated by phytohemagglutin (PHA).

FIG. 13A is a table depicting IC50 and Ki values for various small molecule antagonists identified from a FLIPR® screen using CCR7-transfected CHEM-2 cells.

FIG. 13B is a graph depicting the correlation of Ki values from binding assays and IC50 values from FLIPR® assays for CCR7 antagonists. The goodness of fit, r2, is 0.9616.

FIG. 14A is a graph depicting a FLIPR® agonist assay for CRF1 in CHEM-1 cells using peptide ligands in triplet.

FIG. 14B is a graph and table depicting a CRF1 Dose Response curve and EC50 values for particular CRF1 agonists (sauvagine, oCRF, r/hCRF, hUCN, mUCNII).

FIG. 14C is a graph depicting a FLIPR® agonist assay for CRF2 in CHEM-1 cells using peptide ligands in triplet.

FIG. 14D is a graph and table depicting a CRF2 Dose Response curve and EC50 values for particular CRF1 agonists (sauvagine, oCRF, r/hCRF, hUCN, mUCNII).

FIG. 15A is a graph depicting a comparison of cAMP (left Y axis) and FLIPR® Ca²⁺ (right Y axis) responses for CHEM-1 cells transfected with human CRF1. As depicted in the CHEM-1 expression system, where most of the receptors are forced to couple to the promiscuous Gα₁₅, there is a much greater FLIPR® response (EC50 of 12 nM) than cAMP response (EC50 of 4.9 nM).

FIG. 15B is a graph depicting a comparison of cAMP (left Y axis) and FLIPR® Ca²⁺ (right Y axis) responses for CHO cells transfected with human CRF1. As depicted in the CHO expression system, the cAMP assay yielded a similar EC50 value for the cAMP response (EC50 of 8.4 nM), but did not exhibit Ca flux in CHO cells due to the lack of promiscuous G protein coupling.

FIG. 16A is a schematic showing that GPCRs are now believed to be fluidic, rather than being rigid structures having active (RA) or inactive (R) states.

FIG. 16B is a graph depicting a CRF1 antagonist assay. FLIPR antagonist assays were performed by adding either a peptide antagonist (Astressin) or small molecule antagonist (Compounds 1, Compound 2 or Compound 3) followed by addition of 10 nM of the CRF1 ligand Sauvagine (˜EC50 value). Compound 1 was an allosteric modulator of CRF1 that was identified through FLIPR® High throughput Structure-Activity Relationship (HTSAR) using CRF1/CHEM-1 cells.

FIG. 17A is a graph depicting the orexin dose response in HEK293 cells transfected with the Gq-coupled Orexin Receptor (HCR2) using the pcDNA3 vector.

FIG. 17B is a graph depicting the orexin dose response in CHEM-1 cells transfected with the Gq-coupled Orexin Receptor (HCR2) using the pHS vector. As depicted, the HCR2/pHS/CHEM-1 cell line increased the FLIPR® signal without changing the EC50 of the ligands through endogenous Gα15 in addition to Gq.

FIG. 17C is a graph depicting a GnRH antagonist (Chem-11221) assay showing the correct SAR for Chem-11221.

FIG. 17D is a graph depicting a Gα₁₂-coupled thrombin receptor (PAR1) FLIPR® dose response, which also shows the correct pharmacology in CHEM-1 cell FLIPR® assay.

FIG. 18 is a graph depicting Bmax values for a variety of GPCRs (CXCR1, CXCR2, CXCR3, CXCR4, CCR5, CCR6, CCR7, C3aR and C5aR) expressed using either the pcDNA3.1 vector or the pHS vector. As depicted, all of the GPCRs had a much higher Bmax when expressed using the pHS vector than when expressed using the pcDNA3.1 vector.

### DETAILED DESCRIPTION OF THE INVENTION

The present invention may be readily understood by those skilled in the art by reference to the following descriptions of certain preferred embodiments and examples, whereby the invention may be easily duplicated using readily available means and techniques known to any person skilled in the are. It is understood that these embodiments are provided to clearly describe the instant invention but are not intended to limit the scope and means by which other embodiments of the invention may be conceived or accomplished, or the modifications and variations apparent to anyone skilled in the art.

As broadly described herein, the invention provides compositions and methods for detecting interactions between ligands and their specific GPCRs. Given that many GPCRs have been identified only by homology of captured polynucleotide sequences, numerous of these receptors are not associated with specific natural ligands and are called “orphan” receptors. Methods of the instant invention provide, among other uses, means to identify ligands (e.g., natural ligands) by utility in high throughput screening of compounds and natural molecules. In one embodiment, the invention is an agent that is identified by the screening methods of the invention. Given the importance of GPCRs in drug therapy, agents that are identified as capable of modifying the activity of a particular GPCR could be useful for therapeutic purposes.

In one embodiment, cells are selected that endogenously express the Gα₁₅ and/or Gα₁₆ family of promiscuous G-proteins, and which are particularly compatible with expression of receptors belonging to the GPCR class of receptors. Gα_{q}-coupled receptors can mobilize calcium through coupling to phospholipase C beta 2 (PLCβ2). Although most GPCRs are not linked to this pathway, any GPCR can be linked by means of promiscuous G-proteins, chimeric Gα_{q}-proteins, or in specific cases where GPCRs are intended to provoke calcium flux, by native G-proteins of the Gα_{q} family. However, as described herein, the G-proteins Gα₁₅ and Gα₁₆ are promiscuous, and high levels of endogenous Gα₁₅ and/or Gα₁₆ expression in the cells of the invention provide any GPCR with good linkage to PLCβ and subsequent calcium ion mobilization.

In a particular embodiment, certain features of a novel mammalian GPCR expression vector (e.g., pHS) provide for highly increased expression of recombinant GPCRs on the surface of a cell, and result in successful coupling of the GPCR to the cell's endogenous Gα₁₅ and/or Gα₁₆. As described herein, this results in activation of the PLCβ/calcium pathway upon binding of ligand to the GPCR.

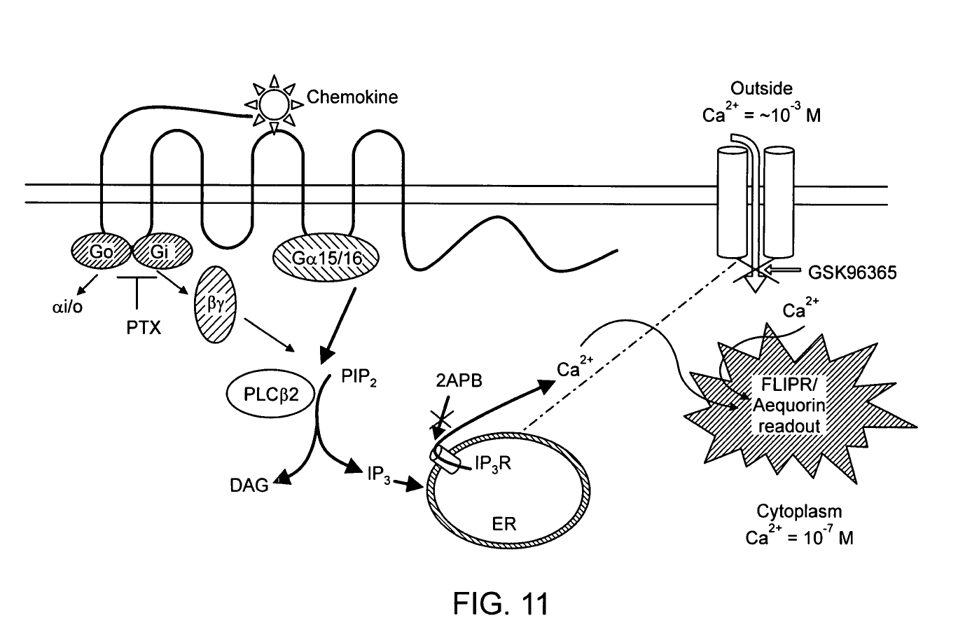

In a further embodiment, cells are provided wherein the calcium-ion related signal produced by GPCR activation is significantly amplified by means of a coordinated activation of calcium release activated calcium channels (CRAC channels). Such channels are opened by the depletion of stored calcium ions, permitting additional calcium ions to enter the cell in a dose-dependent manner (FIG. 11).

Collectively, these features provide a significant improvement over present methods for measuring activation of a GPCR, with increased signal to background ratio and more physiological EC50 values for the ligands than co-transfecting specific types of G proteins (e.g., Gα₁₅, Gα₁₆, Gα_{q} chimeras). As described herein, a conventional method for coupling a non-Gα_{q}-coupled GPCR to the PLCβ/calcium pathway involves co-transfection of a G protein and a GPCR, which can lead to difficulty in maintaining optimal levels of both the GPCR and G-protein and/or can cause constitutive activation of the GPCR thereby increasing background.

GPCRs

All GPCRs operate through a similar molecular mechanism. Activation of GPCR by extracellular stimuli causes conformational changes in the receptor, which results in the intermediate activation of GTP-binding proteins (G proteins) and subunit release. G-proteins are heterotrimeric in nature and are composed of alpha (α), beta (β) and gamma (γ) subunits that are encoded by distinct genes. The alpha subunit is responsible for the binding of GDP and GTP. Binding of a ligand to the GPCR results in a transition of GDP-bound to a GTP-bound a subunit and leads to dissociation of the α-GTP from the βγ dimer. Both α-GTP and the βγ dimer regulate the activities of effectors that transmit the signal to the cell and cell nuclear interior through the production of second messenger molecules (e.g., calcium, cAMP, etc). There are at least 17 G protein genes, and members of G proteins can be grouped into four (or five) main classes, termed Gα_{i/0}, Gα_{q}, Gαₛ and Gα₁₂. As is known in the art, the structural and functional classification of G proteins has been defined by the a subunits (see, e.g., Morris, A. J., and Malbon, C. C. Physiol. Rev. 79(4):1373-1430 (1999); the entire teachings of which are incorporated herein by reference). GPCRs come in many different flavors, and they can couple to a variety of G proteins, thus leading to activation of many signaling pathways upon ligand binding. Most GPCR receptors are linked to a plurality of intracellular signaling pathways by means of one or another member of the Gα family of signaling proteins, generally Gα_{i/0}, Gα_{q}, Gαₛ, and Gα₁₂. Binding of a ligand to a particular GPCR initiates a signal that is detected by measuring some change in properties of some element of the signaling pathway, generally a terminal element, such as ATP, ADP or calcium ion mobilized from storage depots.

As described herein, exogenous nucleic acid sequences that encode one or more GPCRs are introduced into the cells of the invention. As used herein, an exogenous nucleic acid sequence refers to a nucleic acid sequence that does not naturally occur in the cell and/or has been introduced into a cell (e.g., a host cell, a progenitor (ancestor) cell). Any nucleic acid sequence (e.g., DNA, RNA) that can encode a GPCR and that is exogenously expressed can be used in the compositions and methods of the invention. Introduction of exogenous nucleic acid sequences is well known in the art, and can be performed, for example, through transfection. Known transfection methods include, e.g., calcium phosphate precipitation, DEAE dextran-mediated gene transfer, liposome-mediated gene transfer, electroporation, microinjection, retroviral transfection and the use of gene guns. The particular transfection method employed will depend on many factors, including the cell type utilized and GPCR to be produced. For example, calcium phosphate precipitation (described, for example, in Wigler et al., Proc. Natl. Acad. Sci. USA 77:3567 (1980)) and liposome-mediated gene transfer are useful for the transfection of most mammalian cells, but have been found to be more effective with adherent cells. In contrast, electroporation (described, for example, in Potter, et al., Proc. Natl. Acad. Sci. USA 81: 7161 (1988)) is primarily utilized for the transfection of cells in suspension. Retroviral transfection methods are useful for transfection of many mammalian cell types (described for example in Mann, et al., Cell 33:153-159 (1993); Pear, et al., Proc. Natl. Acad. Sci. USA 90(18):8392-96 (1993); and Kitamura, et al., Proc. Natl. Acad. Sci. USA 92:9146-50 (1995)). Various techniques for transfection of mammalian cells are further described in Keown et al., Methods in Enzymology 185:527-37 (1990), Mansour, et al., Nature 336:348-52 (1988) and Sambrook, et al., Molecular Cloning, 2nd Ed., Vol. 3, Chapter 16, particularly sections 68-72 (1989). In one embodiment, an exogenous nucleic acid sequence that encode a GPCR is introduced to a cell as a plasmid or vector.

As described herein, in a particular embodiment, an exogenous nucleic acid sequence that encodes a GPCR is introduced into the cell of the invention. In one embodiment, the GPCR is a chemokine receptor. Chemokine receptors are known to be important immune molecules and represent good targets for drug discovery.

Alternatively, exogenous nucleic acids that, when introduced into the cell, result in high levels of expression of endogenous GPCRs are also encompassed in the methods of the present invention. For example, an exogenous nucleic acid that turns on a normally silent endogenous nucleic acid sequence that encodes a GPCR (or an exogenous nucleic acid that causes high levels of expression of an endogenous nucleic acid sequence that encodes a GPCR) can be introduced into (spliced into the genome) of the cell. Methods for such techniques are known in the art (e.g., U.S. Pat. Nos. 5,641,670, 5,733,761 and 5,733,746, which are incorporated herein by reference).

Cells

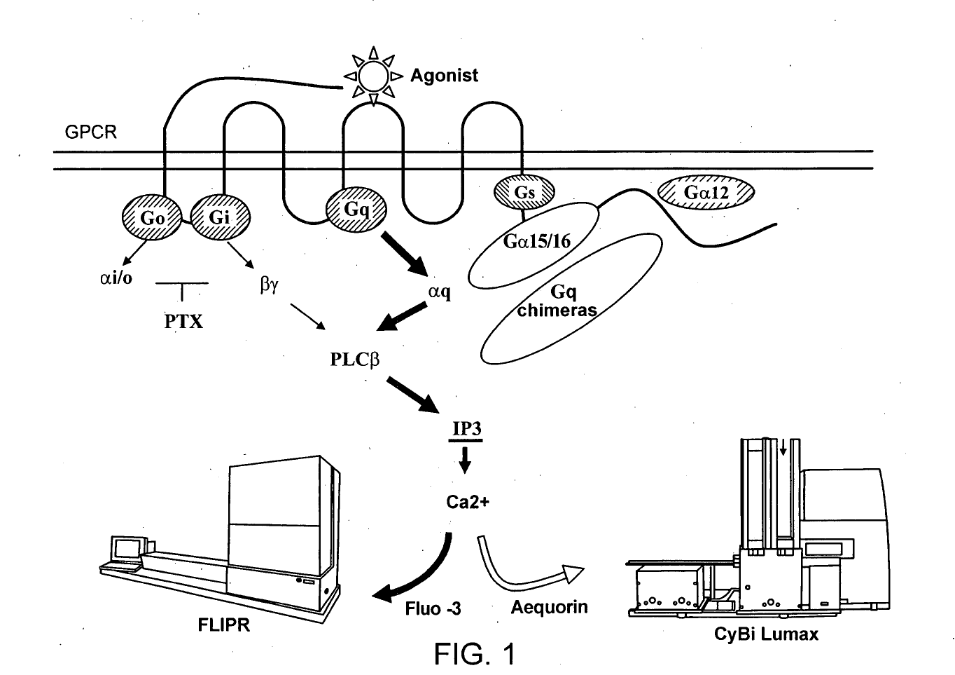

In accordance with the compositions and methods of the invention described broadly herein, this invention, in one aspect, provides for a composition for detection of an activated GPCR (e.g., a Gα_{i/0}-coupled GPCR, a Gα_{q}-coupled GPCR, a Gαₛ-coupled GPCR, a Gα₁₂-coupled GPCR). In a particular embodiment, the inventive composition is comprised of a stable cell line expressing a GPCR and comprising an endogenous promiscuous G-protein, such as Gα₁₅ and/or Gα₁₆, both of which provide GPCRs with linkage to the phospholipase C-beta (PLC-β) pathway, irrespective of the intrinsic linkage of that GPCR by a specific G-protein class to a specific signaling pathway. By coupling Gα₁₅ and/or Gα₁₆ to a GPCR, stimulation of that GPCR by an agonist ligand will cause an increase in intracellular calcium ions through activation of the PLCβ0 pathway (FIGS. 1 and 11). In the compositions and methods of the invention, the Gα₁₅ and/or Gα₁₆ is not introduced into the cell as a recombinant protein(s), by vector or other genetic means, as is the practice and state of the art at this time when seeking high levels of expression. Instead, a panel of cell lines were screened, and, as described herein, particular cell lines (e.g., CHEM-1, CHEM-2, CHEM-3) expressing high levels of an endogenous promiscuous G protein (e.g., Gα₁₅, Gα₁₆) were selected. As used herein, an endogenous promiscuous G-protein refers to a naturally-occurring G-protein (e.g., a G-protein that is naturally produced by a particular cell) that is able to couple and be activated in response to activation by GPCRs of multiple types (e.g., Gα_{i/0}-coupled GPCRs, Gα_{q}-coupled GPCRs, Gαₛ-coupled GPCRs, Gα₁₂-coupled GPCRs) and signal through a particular pathway (e.g., the PLCβ/calcium pathway). To provide cell lines expressing high levels of endogenous Gα₁₅ and/or Gα₁₆, cultured cells were grown and assayed for mRNA and protein expression levels of the promiscuous Gα₁₅ and/or Gα₁₆ G-proteins. The mRNA isolated from the aliquots of the cell cultures was processed and the level of transcription determined by reverse transcriptase polymerase chain reaction (RT-PCR). In addition, western blot analysis was performed to measure Gα₁₅ and/or Gα₁₆ protein expression.

Thus, in one embodiment, the invention is a cell comprising one or more endogenous promiscuous G-proteins and an exogenous nucleic acid sequence that encodes a GPCR, wherein the one or more G-proteins and the GPCR are expressed at high levels.

As described above, an endogenous promiscuous G-protein refers to a naturally-occurring G-protein (e.g., a G-protein that is naturally produced by a particular cell) that is able to couple and be activated in response to activation by GPCRs of multiple types (e.g., Gα_{i/0}-coupled GPCRs, Gα_{q}-coupled GPCRs, Gαₛ-coupled GPCRs and Gα₁₂-coupled GPCRs) and signal through a particular pathway (e.g., the PLCβ/calcium pathway). In a particular embodiment, the endogenous promiscuous G-protein is able to couple and be activated in response to activation by GPCRs of any type, including Gα_{i/0}-coupled GPCRs, Gα_{q}-coupled GPCRs, Gαₛ-coupled GPCRs and Gα₁₂-coupled GPCRs. In another embodiment, the endogenous promiscuous G-protein signals through the PLCβ/calcium pathway. In yet another embodiment, the one or more promiscuous G-protein comprises an a subunit selected from the group consisting of Gα₁₅, Gα₁₆ and a combination thereof.

In the cells of the invention, the endogenous promiscuous G-protein is expressed at a high level. As used herein, “a high level of G-protein expression” or “a G-protein expressed at a high level” refers to at least a 10-fold increase in expression of the G protein as compared to expression of the G protein in a control cell (e.g., a CHO cell).

As described above, an exogenous nucleic acid sequence that encodes a G-protein coupled receptor (GPCR) or results in expression of a GPCR refers to a nucleic acid sequence that does not naturally occur in the cell and/or has been introduced into the cell (e.g., a host cell, a progenitor (ancestor) cell). Any nucleic acid sequence (e.g., DNA, RNA) that encodes a GPCR or causes endogenous GPCRs to be expressed at high levels and that is introduced into the cell (an exogenous nucleic acid sequence) can be used in the compositions and methods of the invention. In one embodiment, the nucleic acid sequence encoding a (GPCR) is present in a plasmid or a vector. In another embodiment, the nucleic acid sequence encoding a G-protein coupled receptor (GPCR) comprises a non-CMV promoter that is operably linked to the GPCR. As used herein, a promoter is “operably-linked” to a GPCR when it is able to control transcription of the GPCR. In yet another embodiment, the nucleic acid sequence encoding a GPCR further comprises an endoplasmic reticulum (ER) export signal. In a particular embodiment, the nucleic acid sequence encoding a G-protein coupled receptor (GPCR) is pHS. pHS, also referred to as pHS vector or pHS plasmid, was deposited on Sep. 20, 2005, on behalf of CHEMICON® International, Inc., 28820 Single Oak Drive, Temecula, Calif. 92590, U.S.A., at the American Type Culture Collection (ATCC), 10801 University Boulevard, Manassas, Va. 20110, U.S.A., under Accession No. PTA-6986.

In the cells of the invention, the GPCR is expressed at a high level. As used herein, “a high level of GPCR expression” or “a GPCR expressed at a high level” refers to expression of a GPCR that is significantly higher than endogenous expression of that GPCR. In one embodiment, such a high level of GPCR expression is greater than about 100,000 receptors/cell. In another embodiment, such a high level of GPCR expression is a Bmax greater than about 1 pmole/mg of protein. Bmax is the top of the saturation binding curve (e.g., in pmol per mg of membrane protein) and is directly proportional to the receptor density. In yet. another embodiment, such a high level of GPCR expression is a greater than about a 10-fold increase in expression of the exogenously-expressed GPCR as compared to the corresponding untransfected cell (i.e., the endogenous level of that GPCR).

The activation of GPCRs coupled to the PLC-β/calcium pathway results in intracellular calcium mobilization, which can be sensed by calcium release activated calcium channel (CRAC channels) that are endogenously highly expressed in particular cells (e.g., certain immune systems cells (e.g., CHEM-2 cells, CHEM-3 cells)). CRAC channels are operated by the intracellular calcium store through the interaction of the IP₃ receptor (IP₃R) on the ER and the CRAC channels. Specifically, CRAC channels sense the calcium concentration in the ER and open when the internal calcium store is depleted. This results in increased calcium rushing into the cell and amplifies the GPCR-mediated calcium mobilization. Such an amplification of the calcium mobilization signal is advantageous for detection, for example, using calcium-sensitive molecules, which include, but are not limited to, calcium-sensitive dyes, calcium-sensitive fluorescent compounds and/or calcium-sensitive detectable proteins. Accordingly, in one embodiment, the cell further comprises an endogenous calcium release activated calcium (CRAC) channel. In another embodiment, the cell takes up extracellular calcium through the CRAC channel.

As described herein, generally it is only GPCRs that associate with G proteins of the Gα_{q} family that signal through the PLCβ/calcium pathway. Thus, in one embodiment, the exogenously-introduced GPCR binds to a G protein of the Gα_{q} family. An advantage of the cells and expression systems described herein is that they are able to couple a GPCR (including non-Gα_{q}-associated GPCRs) to the PLCβ/calcium pathway. Accordingly, in one embodiment, the exogenously-introduced GPCR binds to a G protein selected from the group consisting of a G protein of the Gα_{i/0} family, a G protein of the Gαₛ family and a G protein of the Gα₁₂ family.

The cells and methods of the invention are designed to couple GPCRs (including non-Gα_{q}-associated GPCRs) to the PLCβ/calcium pathway. Thus, in one embodiment, the cell exhibits an increase in intracellular free calcium in response to binding of one or more ligands to the GPCR.

As demonstrated by the screening of multiple cell lines, particular cells have the desired characteristics of comprising one or more endogenous promiscuous G-proteins that are expressed at a high level, and further expressing (capable of expressing) an exogenous nucleic acid sequence encoding a GPCR at a high level. In one embodiment, the cell of the invention is a mammalian cell. In particular embodiments, the cell of the invention is a human cell or a murine cell. In yet another embodiment, the cell of the invention is selected from the group consisting of:
i) a CHEM-1 (RBL-2H3) cell, deposited at the American Type Culture Collection (ATCC) as Accession Number CRL-2256;
ii) a CHEM-2 (U937) cell, deposited at the American Type Culture Collection (ATCC) as Accession Number CRL-1593.2; and
iii) a CHEM-3 (BA/F3) cell, deposited at the Deutsche Sammlung von Mikroorganismen und Zellkuturen GmbH (DSMZ) as Accession Number ACC300.
   In a particular embodiment, the cell is an isolated cell. As used herein, a composition (e.g., a cell, a plasmid) is isolated (pure, substantially pure) when it is substantially free of cellular material, when it is isolated from non-recombinant cells, or free of chemical precursors or other chemicals when it is chemically synthesized.

As described herein, the cells of the invention express endogenous promiscuous G proteins and an exogenous nucleic acid sequence that encodes (or causes to be expressed) one or more GPCRs at a high level. In one embodiment, such cells are amenable to introduction of exogenous nucleic acid encoding the GPCR (e.g., a cell that is easy to transfect). In one embodiment, the cell has a transfection efficiency of >50% (e.g., as measured using a GFP construct). In another embodiment, such are cells are easy to culture. In one embodiment, the cell has a doubling time that is 24 hours or less. In another embodiment, the cell of the invention endogenously expresses a (one or more) GPCR at a low level (e.g., less than 1000 GPCRs per cell). Low endogenous expression of a GPCR of interest results in less background signaling and can therefore be advantageous in the methods of the invention.

In one embodiment, the cell of the invention is an adherent cell. Adherent cells (e.g., CHEM-1 cells) adhere to substrates (e.g., the bottom of plates), and can be more easily assayed using assays or detection methods that detect a signal (e.g., calcium) present at or near the bottom of a vessel (e.g., a microtiter plate) in which an assay takes place (e.g., a FLIP® assay). In another embodiment, the cell of the invention is a non-adherent cell, which remains in suspension (e.g., CHEM-2 cells, CHEM-3 cells). For assays that detect a signal (e.g., calcium) present at or near the top of a vessel (e.g., a microtiter plate) in which an assay takes place (e.g., an aequorin assay), such cells can be more accurately assayed.

In one embodiment, the cell of the invention further comprises a (one or more) calcium-sensitive molecule. Inclusion of a calcium-sensitive molecule can aid in detecting and assaying calcium mobilization. Calcium-sensitive molecules are known in the art, and include, but are not limited to, calcium-sensitive dyes, calcium-sensitive fluorescent compounds and/or calcium-sensitive detectable proteins. In one embodiment, the calcium-sensitive molecule is a calcium-sensitive dye (e.g., Fluo-3, Fluo-4, Indo-1, Fura-2, Rhod-2, Oregon green and calcium green-2). In another embodiment, the calcium-sensitive molecule is a calcium-sensitive detectable protein (e.g., a bioluminescent protein). In a particular embodiment, the calcium-sensitive detectable protein is a bioluminescent protein selected from the group consisting of luciferase, aequorin, apo-aequorin and a derivative or mutant of any of the foregoing. Such calcium-sensitive detectable proteins can also be encoded by a nucleic acid sequence. Any nucleic acid (e.g., DNA, RNA) that encodes a calcium-sensitive detectable protein is encompassed by the invention. In a particular embodiment, the nucleic acid sequence encoding a calcium-sensitive detectable protein is present in a plasmid or vector. In another embodiment, the nucleic acid sequence encoding the calcium-sensitive detectable protein and the nucleic acid sequence encoding the GPCR are both present in a plasmid. In still another embodiment, the nucleic acid sequence encoding the calcium-sensitive detectable protein and the nucleic acid sequence encoding the GPCR are present in the same plasmid.

Plasmids

In one embodiment, the invention is a plasmid or vector comprising a nucleic acid sequence encoding a (one or more) GPCR, wherein the GPCR is expressed at a high level. In another embodiment, the plasmid comprises a nucleic acid sequence. encoding a G-protein coupled receptor (GPCR), wherein the nucleic acid sequence encoding the GPCR further comprises a weak promoter (e.g., a non-CMV promoter) that is operably linked to the GPCR. As demonstrated herein, the presence of a weak promoter (e.g., the SFFV LTR promoter contained in the pHS vector) in a plasmid of the invention, when transfected into the cells described herein, results in high levels of expression of the exogenously-introduced GPCR. In another embodiment, the plasmid comprises a nucleic acid sequence encoding a G-protein coupled receptor (GPCR), wherein the nucleic acid sequence encoding the GPCR further comprises an endoplasmic reticulum (ER) export signal.

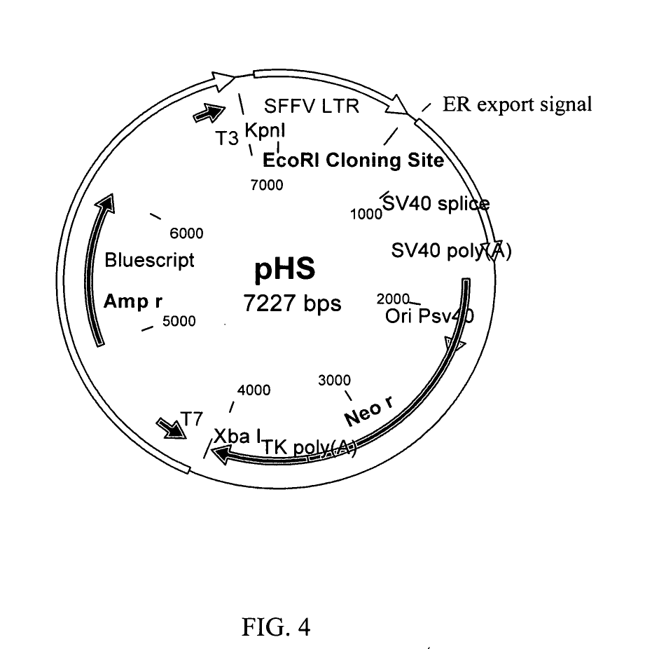

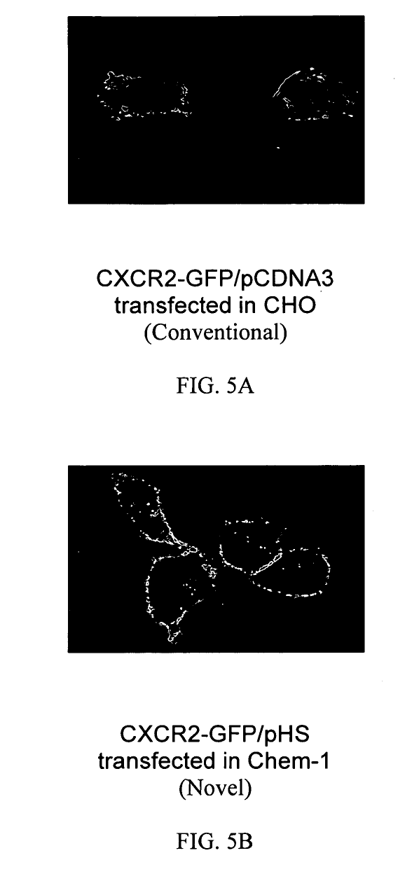

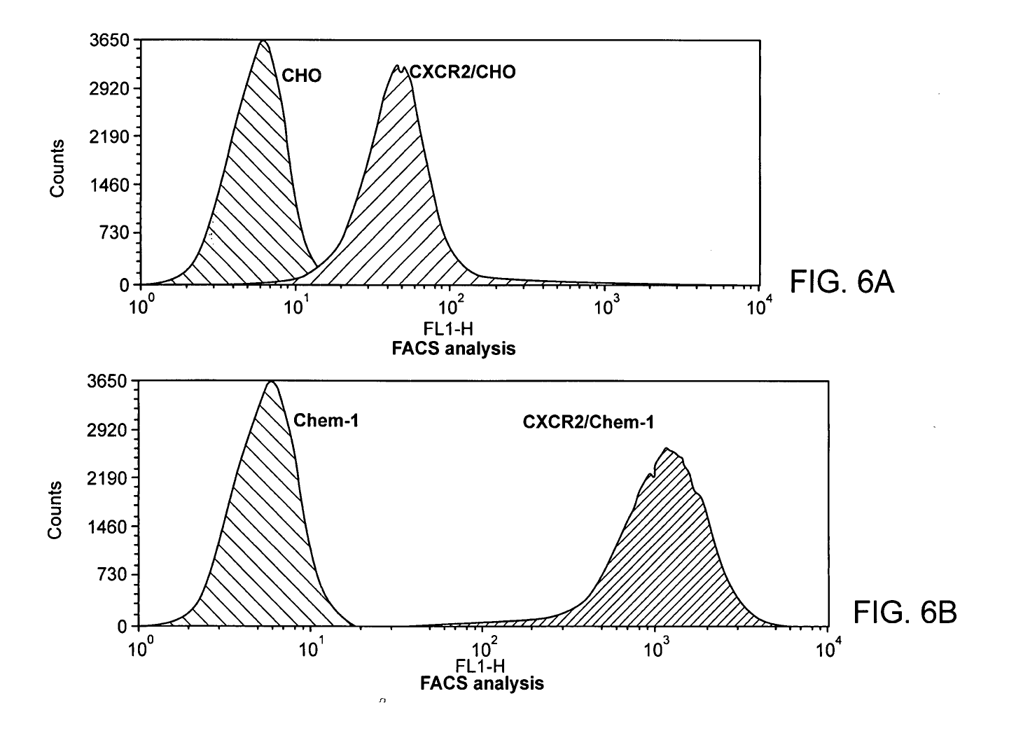

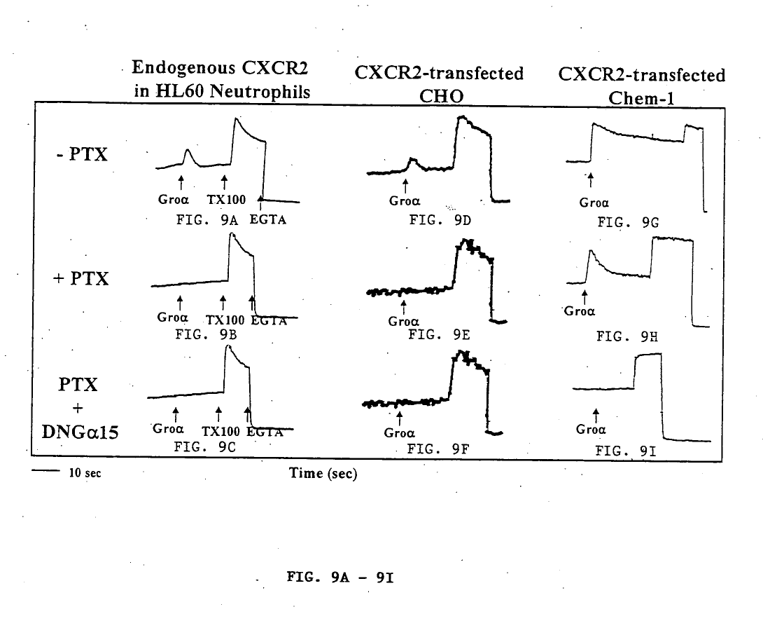

As described herein, a novel mammalian expression vector, pHS, which was deposited at the American Type Culture Collection (ATCC) as Accession Number PTA-6986, was developed. pHS contains a non-CMV promoter (SFFV LTR) and an ER export signal (FIG. 4), and is compatible with the cells of the invention (e.g., CHEM-1, CHEM-2 and CHEM-3 cell lines). Further, as demonstrated herein, pHS reduced ER retention of GPCRs, resulted in a significant increase in the level of functional receptor expression on the cell surface (FIGS. 5 and 6) and subsequent functional coupling to the endogenous promiscuous G-protein, and resulted in superior calcium mobilization and an increased FLIPR® signal (FIG. 9). Accordingly, in one embodiment, the invention is pHS vector, deposited at the American Type Culture Collection (ATCC) as Accession Number PTA-6986. In another embodiment, the invention is a cell transfected with pHS vector. Suitable cells include any cell that can be transfected with pHS.

Methods of Screening, Methods of Expressing GPCRs and Methods of Measuring Alterations in Intracellular Calcium Levels

As described herein, over 60% of current prescription drugs are targeted toward GPCRs, and, as such, GPCRs are an important focus for drug discovery research. Further, as described herein, the cells of the invention are well suited for screening for agents that modify the activity of a GPCR. Accordingly, in particular embodiments, the invention is a method of screening for agents that modify the activity and/or expression of a GPCR.

In one embodiment, the invention is a method of identifying an agent that modulates the activity of a G-protein coupled receptor (GPCR) or an agent that activates the GPCR. In the method, a cell that comprises one or more endogenous promiscuous G-proteins and an exogenous nucleic acid sequence that encodes a G-protein coupled receptor (GPCR), wherein the one or more G-proteins and the GPCR are expressed at high levels, an agent that activates the GPCR, and a test agent are combined, and the activity of the GPCR is detected. An alteration in activity of the GPCR, relative to a control, indicates that the test agent modulates activity of the GPCR or an agent that activates the GPCR.

In one embodiment, the test agent decreases activity of the GPCR. In another embodiment, the test agent increases activity of the GPCR. For example, the test agent can be a ligand or agonist that competes with the agent that activates the GPCR used in the assay for binding and/or activation of the GPCR.

In another embodiment, the invention is a method of identifying an agent that increases activity of a GPCR. The method comprises combining a cell that comprises one or more endogenous promiscuous G-proteins and an exogenous nucleic acid sequence that encodes a G-protein coupled receptor (GPCR), wherein the one or more G-proteins and the GPCR are expressed at high levels, and a test agent, and detecting activity of the GPCR. An increase in activity of the GPCR, relative to a control, indicates that the test agent increases activity of the GPCR.

In one embodiment, the agent that increases activity of a GPCR is an agonist. As used herein, an agonist is an agent that increases activity of a GPCR, either directly or indirectly. In a particular embodiment, the agent that increases activity of a GPCR is an agonist that binds and interacts directly with the GPCR. In another embodiment, the agent that increases activity of a GPCR is a ligand (e.g., a naturally-occurring ligand, a non-naturally occurring ligand).

In a particular embodiment, the invention is a method of identifying a ligand of a G-protein coupled receptor (GPCR). In the method, a cell that comprises one or more endogenous promiscuous G-proteins and an exogenous nucleic acid sequence that encodes a G-protein coupled receptor (GPCR), wherein the one or more G-proteins and the GPCR are expressed at high levels, and a test ligand are combined, and activity of the GPCR is detected. An increase in activity of the GPCR, relative to a control, indicates that the test ligand is a ligand of the GPCR. In a particular embodiment, the GPCR is an orphan GPCR. Thus, the cells of the invention, because of their desirable properties, allow for a method of deorphaning an orphan GPCR.

As described, the cells of the invention couple GPCRs (e.g., Gα_{i/0}-coupled GPCRs, Gα_{q}-coupled GPCRs, Gαₛ-coupled GPCRs, Gα₁₂-coupled GPCRs) to the PLCβ/calcium pathway. Thus, in one embodiment, the method of identifying an agent that modifies activity (e.g., increases activity, decreases activity) of a GPCR is identified by an alteration (e.g., an increase, a decrease) in intracellular free calcium. In a particular embodiment, the alteration (e.g., an increase, a decrease) in intracellular free calcium is detected using a Fluorometric Imaging Plate Reader (FLIPR®) or aequorin technology. Other techniques (e.g., electrophysiology) for measuring alterations in intracellular free calcium are known to those of skill in the art.

FLIPR® and aequorin technologies have become the systems of choice for measuring the changes in intracellular calcium in a high throughput manner. They both provide rapid and sensitive read-out for many GPCR drug targets. However, as described herein, not all GPCRs couple to Gα_{q} and activate the PLCβ pathway leading to calcium mobilization. As a much more convenient, reproducible and highly sensitive alternative to the current methods of co-transfecting with promiscuous and/or chimeric G-proteins to promote a FLIPR® response, the cells of the invention couple GPCRs to the PLCβ/calcium pathway, and thus are well suited for the screening methods described herein.

In the screening methods described herein, alterations in activity of the GPCR are determined relative to a control. A suitable control is to perform the assay in the absence of the test agent. In addition, a control can be a standard control that is established using a large number of samples and a statistical model to obtain a standard value or control value. Other suitable controls are readily apparent to those of skill in the art.

A variety of test agents or test compounds, including, but not limited to, proteins (e.g., antibodies), peptides, peptidomimetics, small organic molecules, nucleic acids and the like, can be assayed in the methods of the invention. Such test agents can be individually screened or one or more test agents can be assayed simultaneously. Where a mixture of test agents is assayed, the test agents selected by the methods described can be separated (as appropriate) and identified using suitable methods (e.g., sequencing, chromatography, etc.). The presence of one or more agents (e.g., a ligand, an inhibitor, a promoter) in a test sample can also be determined according to these methods.

Test agents that modify the activity of a GPCR can be identified, for example, by screening libraries or collections of molecules, such as, the Chemical Repository of the National Cancer Institute, using the methods described herein. Libraries, such as combinatorial libraries, of compounds (e.g., organic compounds, recombinant or synthetic peptides, “peptoids”, nucleic acids) produced by combinatorial chemical synthesis or other methods can be tested (see e.g., Zuckerman, R. N. et al., J. Med. Chem., 37: 2678-2685 (1994) and references cited therein; see also, Ohlmeyer, M. H. J. et al., Proc. Natl. Acad. Sci. USA 90:10922-10926 (1993) and DeWitt, S. H. et al., Proc. Natl. Acad. Sci. USA 90:6909-6913 (1993), relating to tagged compounds; Rutter, W. J. et al. U.S. Pat. No. 5,010,175; Huebner, V. D. et al., U.S. Pat. No. 5,182,366; and Geysen, H. M., U.S. Pat. No. 4,833,092). Where compounds selected from a library carry unique tags, identification of individual compounds by chromatographic methods is possible.

In one embodiment, the invention is an agent that is identified by the screening methods of the invention. Given the importance of GPCRs in drug therapy, agents that are identified as capable of modifying the activity of a particular GPCR could be useful for therapeutic purposes.

The invention is also directed to methods of expressing a G-protein coupled receptor (GPCR) in a cell, comprising transfecting the cell with a nucleic acid sequence encoding the GPCR, wherein the cell comprises one or more endogenous promiscuous G-proteins, and wherein the one or more G-proteins and the GPCR are expressed at high levels. In a particular embodiment, the nucleic acid sequence that encodes the GPCR is present in the pHS vector.

As described herein, the cells of the invention couple GPCRs to the PLCβ/calcium pathway. Therefore, in one embodiment, the invention is a method of measuring an alteration in intracellular calcium in a cell. In the method, a cell that comprises one or more endogenous promiscuous G-proteins and an exogenous nucleic acid sequence that encodes a G-protein coupled receptor (GPCR), wherein the one or more G-proteins and the GPCR are expressed at high levels, is combined with an agent that activates the GPCR, and intracellular calcium in the cell is measured.

In another embodiment, the invention is a method of coupling a G-protein coupled receptor (GPCR) to the PLCβ pathway. The method comprises combining a cell that comprises one or more endogenous promiscuous G-proteins and an exogenous nucleic acid sequence that encodes a G-protein coupled receptor (GPCR) with an agent that activates the GPCR, under conditions in which the GPCR is activated. In the method, the one or more G-proteins and the GPCR are expressed at high levels, and the one- or more G-proteins are selected from the group consisting of a G protein of the Gα_{i/0}/family, a G protein of the Gαₛ family and a G protein of the Gα₁₂ family.

As described herein, the invention encompasses cells comprising receptors that, when stimulated by a ligand (or “agonist”), can effect a mobilization of calcium ions from intracellular storage or sequestration depots, such that the mobilized calcium ion flux may be demonstrated by various analytical means, including, but not limited to, FLIPR® and aequorin analysis. Additionally, the invention can detect and identify those agents and test substances that will modulate (e.g., increase, decrease) activity of a receptor. Without limiting the broad scope of the invention, one particular use of the invention is directed to the detection of ligand binding by the class of receptors referred to as GPCRs.

The preferred embodiments and the examples shown are understood not to limit the scope of the numerous conceptions easily envisioned and derived from the inventive entity. That is, the invention is not directed only to the specific receptors or ligands provided in the Examples, but rather the invention encompasses all receptors and ligands for which the conception can be applied, and likewise the principle of combining a calibrated G-protein coupled receptor (GPCR) expression with a strong endogenous expression of promiscuous G-proteins. In particular, this invention is not limited by the embodiments, examples or terminology used herein, to any particular GPCR, irrespective of whether the function(s) or ligand(s) of the receptor is/are known or are not known (hereafter an “orphan” GPCR”). There is likewise no limit to the use of the invention to detect, measure or modify any part of a cellular or in vitro signaling pathway affecting the level or disposition of calcium ions in cells, or specific conditions, specific methods, specific genes, specific cell types, or any specific gene or protein sequences, or specific drugs or agonists or antagonists, which have utility in the invention. It is also understood that the terminology used herein is for the purpose of describing the invention and must not be taken to be limiting of the scope of the invention.

### EXAMPLES

Methods & Materials

Expression Plasmids, Cell Lines and Antibodies

The expression plasmid, pHS, which was used for expression of GPCRs is based on Stratagene's pBluescript® backbone (Stratagene, La Jolla, Calif.). A schematic of pHS is provided as FIG. 4. In addition, the sequence of relevant portions of pHS (e.g., the SFFV LTR promoter, the ER export signal) are described herein. pHS, also referred to as pHS vector or pHS plasmid, was deposited on Sep. 20, 2005, on behalf of CHEMICON® International, Inc., 28820 Single Oak Drive, Temecula, Calif. 92590, U.S.A., at the American Type Culture Collection (ATCC), 10801 University Boulevard, Manassas, Va. 20110, U.S.A., under Accession No. PTA-6986.

Other expression plasmids, such as pcDNA3 and pCDNA5FRT, which were used for comparison purposes, were purchased from InVitrogen (Carlsbad, Calif.).

All of the mammalian cell lines that were used for promiscuous G protein screening and expression of recombinant GPCRs were either purchased from ATCC or obtained from the CHEMICON® cell bank. The cDNAs for GPCRs and G proteins were cloned by RT-PCR or PCR using primers according to the public domain. Specifically, the primers that were used are based on the homologous sequence between Gα₁₅ and Gα₁₆ at the N-terminus and C-terminus. The forward oligonucleotide primer that was used was 5′ atg gcc cgg tcc ctg ac 3′ (SEQ ID NO:1) and the reverse oligonucleotide primer was 5′ gtc aca gca ggt tga tct 3′ (SEQ ID NO:2). Antibodies that were used for FACSorting and analysis were obtained from CHEMICON's catalog. The QuikChange mutagenesis kit was purchased from Stratagene (La Jolla, Calif.).

Cell Transfection

Cells were transfected using Lipofectamine (InVitrogen, Carlsbad, Calif.) or electroporation (BioRad, Hercules, Calif.) according to the manufacturer's instructions. Stable pools were selected using the appropriate antibiotics and FACSorted to single cell clone using specified antibodies to GPCRs. Dye-conjugated or unconjugated monoclonal antibodies (mAbs) or rabbit antisera against various GPCRs were obtained either from commercial sources (e.g., BD Biosciences, San Jose, Calif.; R&D Systems, Minneapolis, Minn.) or from CHEMICON® International, Inc. (Temecula, Calif.). For secondary staining, dye-conjugated purified Fab fragments with the relevant species-specific reactivity were obtained from commercial sources (Molecular Probes/InVitrogen, Carlsbad, Calif.).

Membrane Preparations

Crude membrane fractions were prepared from stably transfected cell by N₂ cavitation and differential centrifugation, and stored at −80° C. in 50 mM Tris Cl with 10% glycerol and 1% BSA.

Receptor Binding Assays

1-5 μg/well of crude membrane protein was thawed and combined with 50,000 to 100,000 cpm of radioligand (2200 Ci/mmol) and cold competitors in assay buffer (50 mM Hepes, 5 mM MgCl₂, 1 mM CaCl₂, 0.2% BSA) for 2 hours at 25° C. Reactions were performed in 96-well GF/C glass fiber filter plates (PerkinElmer, Cat. No. 6005174, Wellesley, Mass.). Binding was terminated by adding 100 μl/well of wash buffer (50 mM Hepes, pH 7.4, 500 mM NaCl, 0.1% BSA) and aspiration through the plate on a cell harvester, followed by three 0.2 ml washes with wash buffer. The filter plate was then dried and counted on a Microbeta liquid scintillation counter (PerkinElmer, Wellesley, Mass.).

For example, the radioligand saturation binding assays of CXCR2 were performed in a 96-well plate by incubating 5 μg of GPCR membrane preparation with increasing concentrations (0˜2 nM) of ¹²⁵I labeled ligand Groα (PerkinElmer, Wellesley, Mass.), in the absence and presence of access amount of cold ligand Groα.

Intracellular [Ca²⁺] Measurements and FLIPR® Assay

Cells (50,000/well) were seeded in a 96 well FLIPR® plate (clear bottom, black wall TC plate) the day before the experiment, and then loaded with fluorescent calcium dye Fluo-4 or Indo-1 (Molecular Probes) and incubated for 30 minutes at 37° C. in the dark with Pluronic F-127 and Probenecid in HBSS buffer (with Ca²⁺ and Mg²⁺). The cells were then washed twice with HBSS and read for calcium mobilization after the ligand addition on FLEX Station or FLIPR®. The loading efficiency and integrity of the Ca²⁺ flux assay were assessed by comparing the signaling response to ionomycin treatment before and after each run.

FLIPR® (Fluorometric Imaging Plate Reader) is no-doubt the most widely used High Throughput Screening platform in today's drug discovery industry. It is designed for cell-based fluorescence assays to monitor changes in intracellular Ca flux, intracellular pH and sodium, and membrane potential, and generates highly sensitive, real-time kinetic data information. It consists of a versatile 6-position platform and integrated 384 or 96 well pipettor, excitation and emission optics, argon laser and cooled CCD camera to simultaneously capture the emitted signal from the entire plate in 1 second. An integrated plate stacker and stacker stage and integrated tip washer can allow the FLIPR® to run the assay in an almost hands-free mode, and can potentially increase the throughput to 100 plates/day, with 384 format, allowing up to 40,000 compounds to be screened per day. It also can reduce the reagent cost to about 1 penny per well, as compared to about $1/well for a cAMP assay.

Chemotaxis Assays

Cells were suspended in HBSS buffer (with Ca²⁺ and Mg²⁺) containing 1% fetal calf serum (FCS), and treated with specified chemokines (R&D Systems, Minneapolis, Minn.) for 2 hours at 37° C., or left untreated. Chemotaxis assays were performed using CHEMICON's transmigration kit (Catalog No. ECM580; Temecula, Calif.).

FACS Analysis

FACS (Florescent Activated Cell) Analysis was performed using a Becton Dickinson Flow Cytometer to detect cell surface receptor expression of a GPCR. In particular, cells were harvested, washed and incubated with a PE (phycoerythin)-conjugated anti-GPCR antibody (e.g., PE-conjugated anti-CXCR2), and then assayed for staining using a FACStar instrument (Becton Dickinson, Franklin Lakes, N.J.), with a nozzle size of 80 microns.

Results

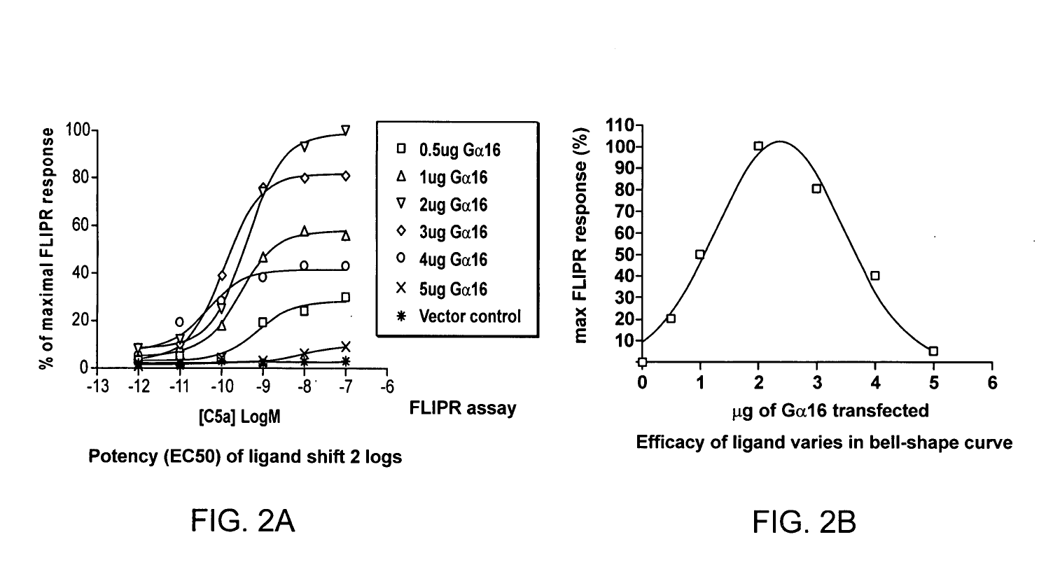

The experiments described herein demonstrate that it is possible to funnel all GPCR signaling pathways such that they result in a common and simple read-out, namely an increase in intracellular free calcium in response to binding of a ligand to the GPCR. As depicted in FIG. 1, a conventional method for coupling non-Gα_{q}-coupled receptors to the PLCβ/calcium pathway is to co-transfect either a promiscuous G protein (e.g., Gα₁₅, Gα₁₆) or Gα_{q} chimeras to promote FLIPR® or Aequorin readout (FIG. 1). However, there are limitations and drawbacks to this conventional method. In particular, it is difficult to control and normalize the amount of G protein that is transfected, which often causes a significant shift of the ligand EC50 and a change in the structure-activity relationship (SAR) (see, e.g., FIGS. 2A and 2B). As depicted in FIGS. 2A and 2B, the absolute amount of Gα₁₆ is critical to generate the correct potency and efficacy of the ligand. In addition, overexpression of recombinant Gα₁₅ and/or Gα₁₆ or Gq chimeras can cause constitutive activation of GPCRs, which can generate high background.

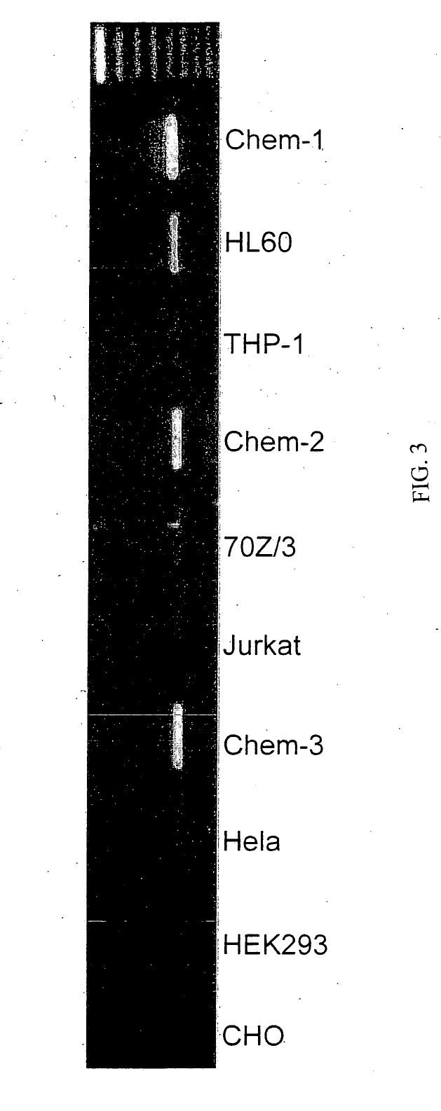

In order to avoid the problems associated with co-transfection of Gα₁₅, Gα₁₆ and/or Gα_{q} chimeras to promote FLIPR® or aequorin responses for GPCRs, RT-PCR and western blot analysis using primers or antibodies common for both Gα₁₅ and Gα₁₆ were performed to identify cells with endogenous Gα15 and/or Gα16 expression (FIG. 3). An example of using RT-PCR to analyze expression of particular cell lines is depicted in FIG. 3. As shown, particular cell lines (e.g., CHEM-1 (RBL-2H3 cells), CHEM-2 (U937 cells), CHEM-3 (BA/F3 cells), HL60) have high levels of endogenous Gα₁₅ and/or Gα₁₆ expression. Although HL60 exhibited a high level of endogenous Gα₁₅ and/or Gα₁₆ expression, subsequent experiments demonstrates that this cell line was difficult to transfect.

Unfortunately, the commonly-used CMV promoter, which is used in most mammalian expression vectors, such as pcDNA3, is silenced in the host cell lines described herein and results in little or no recombinant GPCR expression. Accordingly, a novel mammalian expression vector, pHS, which is deposited at the American Type Culture Collection (ATCC) as Accession Number PTA-6986, was developed. FIG. 4 is a schematic of the pHS expression vector. pHS contains a non-CMV promoter (SFFV LTR) and an ER export signal (FEYEFNEVEF; SEQ ID NO:3) (FIG. 4), and is compatible with the CHEM-1, CHEM-2 and CHEM-3 cell lines that exhibit high levels of endogenous Gα₁₅ and/or Gα₁₆ expression. The non-CMV promoter (SFFV LTR promoter) that is contained in the pHS vector has at least two important improved features for GPCR expression. A first feature is that it is compatible with the cell lines described herein (e.g., CHEM-1, CHEM-2, CHEM-3), such that it highly expresses the exogenous nucleic acids of interest (e.g., GPCRs). A second feature is that the non-CMV promoter results in a rate of transcription that is not too fast to overwhelm the chaperon system and thereby cause ER retention of the expressed protein. Accordingly, this promoter is able to deliver more GPCRs to the cell surface. Experimentation using many viral promoters revealed that the Spleen Focus Forming Virus (SFFV) LTR, which is encoded by the pHS vector, was an excellent promoter for GPCR expression in this system. The sequence of SFFV LTR is:

(SEQ ID NO:4)

tgcagacacaacactgctctacgggcatgagtggccaccttccatttttt
ttttaagtgtgtgtgctgacgtcacaagaactcaggtactcttacttcct
acatggtacgagttcttaccacttagccatttcttcatcctgaaagaccc
caccaagttgcttagcctgatagccgcagtaacgccattttgcaaggcat
ggaaaaataccaaaccaagaatagggaagttcagatcaagggcgggtaca
cgaaaacagctaacgttgggccaaacaagatatctgcggtaagcagtttc
ggccccggcccggggccaagaacagatggtccccagatatggcccaaccc
tcagcagtttcttaagacccatcagatgtttccaggctcccccaaggacc
tgaaatgaccctgtgccttatttgaattaaccaatcagcccgcttctcgc
ttctgttcgcgcgcttttgcttcccgagctctataaaagagctcacaacc
cctcactcggcgcgccagtcctccgacagactgagtcgcccgggtacccg
tgttcccaataaagcctcttgctgattgcatccgaatcgtggactcgctg
atccttgggagggtctcctcagattgattgactgcccacctcgggggtct
ttcatttgggggctcgtccgggatttggagacccccgcccagggaccacc
gacccaccgtcgggaggtaagctggccagcgatcgttttgtctccgtctc
tgtctttg.

The ER export signal (FEYEFNEVEF; SEQ ID NO:3) encoded by the pHS vector is a 9 amino acid hydrophobic motif at the carboxyl-terminal. It has been shown to increase GPCR surface expression by two-fold.

pHS reduced ER retention of GPCRs, resulted in a significant increase in the level of functional receptor expression on the cell surface (FIGS. 5 and 6) and subsequent functional coupling to the endogenous promiscuous G-protein, and resulted in superior calcium mobilization and an increased FLIPR® signal (FIG. 9). While not wishing to be bound to any particular theory, it is believed that exogenous proteins that are expressed under the control of the CMV promoter, because it is a very powerful promoter, often overwhelm the chaperon system of protein expression and cause ER retention of the protein, particularly for large transmembrane proteins, such as the seven transmembrane GPCRs. Selection of a weaker promoter, such as the SFFV LTR promoter of the pHS vector, resolves the ER retention problem, as well as the host cell compatibility problem (e.g., overcoming the silencing effect observed with pcDNA3-expressed GPCRs in CHEM-1, CHEM-2 and CHEM-3 cells).

As depicted in FIGS. 5A, 5B, 6A and 6B, expression of a green fluorescent protein (GFP)-labeled CXCR2 GPCR (CXCR2-GFP) using the pHS vector and CHEM-1 cells resulted in greater cell surface expression than did the CMV promoter-containing pcDNA3 vector and CHO cells. Specifically, GFP was fused to the C-terminus of CXCR2 and transfected into CHO and CHEM-1 cells using different compatible expression vectors (either pcDNA3 or pHS) and fluorescent imaging and FACS analysis were performed. The fluorescent imaging (compare FIG. 5A and FIG. 5B) and FACS analysis (compare FIG. 6A and FIG. 6B) of CXCR2-GFP expression clearly demonstrate greater expression of the GPCR on the cell surface using the pHS vector and CHEM-1 cells. The CXCR2-GFP fusion protein used in these experiments contained the entire coding sequence of CXCR2 (except the stop codon; GenBank Accession No. M73969) fused to CHEMICON's GFP sequence. This sequence is as follows:

(SEQ ID NO:5)

ATGGCCAASCAGATCCTGAAGAACACCTGCCTGCAGGAAGTGATGAGCTA
CAAGGTCAACCTGGAGGGCATCGTGAACAACCACGTCTTTACCATGGAGG
GCTGCGGCAAGGGCAACATCCTGTTCGGCAACCAATTGGTGCAGATCCGC
GTGACCAAGGGCGCCCCCCTGCCCTTCGCCTTCGACATCGTGAGCCCCGC
CTTCCAGTACGGCAACCGTACGTTCACAAAGTACCCCAACGACATCAGCG
ACTACTTCATCCAGAGCTTCCCCGCCGGCTTCATGTACGAGCGCACCCTG
CGCTACGAGGACGGCGGCCTGGTGGAGATCCGCAGCGACATCAACCTGAT
CGAGGACAAGTTCGTGTACCGCGTGGAGTACAAGGGCAGCAACTTCCCCG
ACGACGGGCCCGTGATGCAGAAGACCATCCTGGGCATCGAGCCCAGCTTC
GAGGCCATGTACATGAACAACGGCGTGCTGGTGGGCGAGGTCATCCTGGT
GTACAAGCTGAACAGCGGCAAGTACTACAGCTGCCACATGAAGACCCTGA
TGAAGAGCAAGGGCGTGGTCAAGGAGTTCCCCAGCTACCACTTCATCCAG
CACCGCCTGGAGAAGACCTACGTGGAGGACGGCGGCTTCGTGGAGCAGCA
CGAGACCGCCATCGCCCAGATGACCAGCATCGGCAAGCCCCTGGGATCTC
TGCACGAGTGGGTGTARACCCGCTGATCAGCCTCGACTGTGCCTTCTAGT
TGCCAGCCATCTGTTGTTTGCCCCTCCCCCGTGCCTTCCTTGACCCTGGA
AGGTGCCACTCCCACTGTCCTTTCCTAATAAAATGA

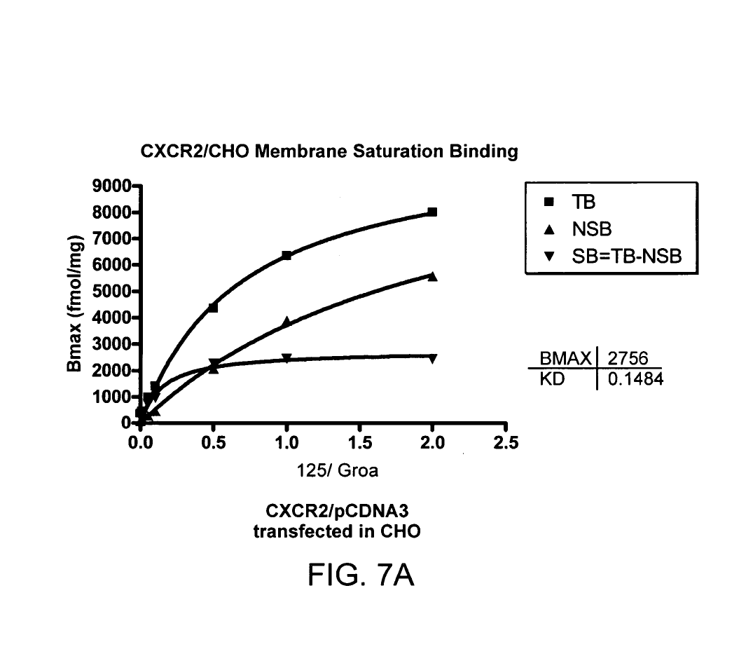

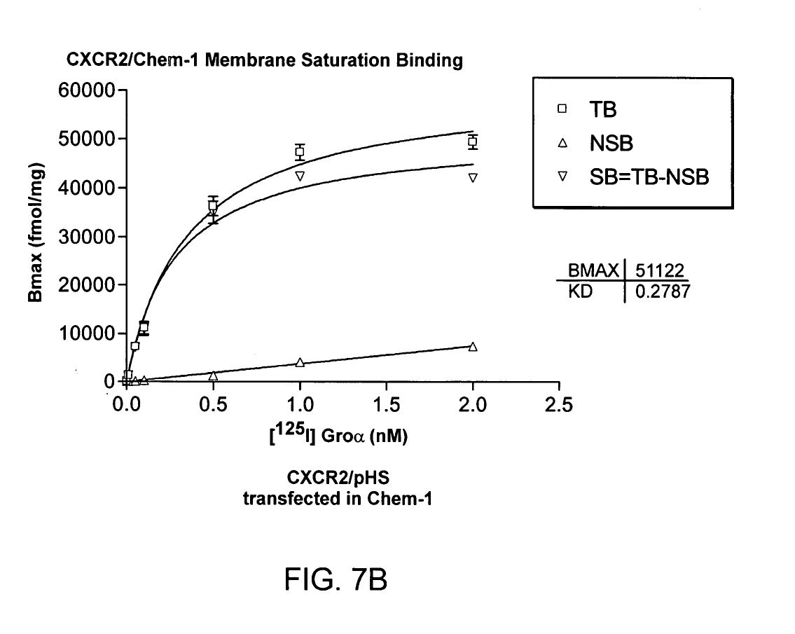

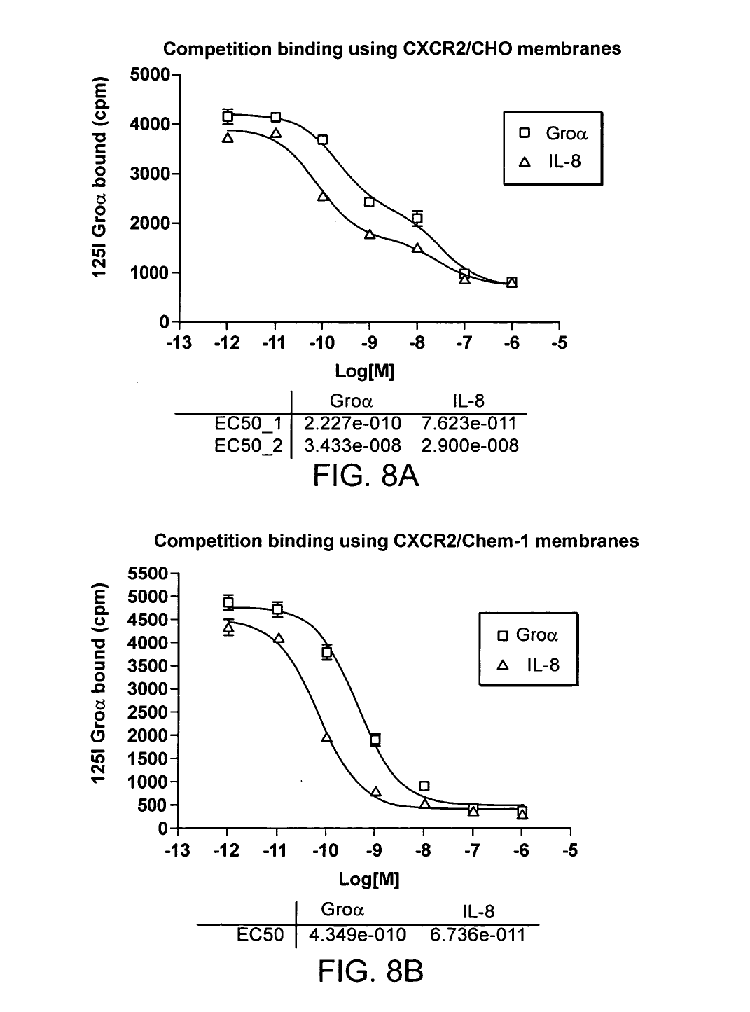

The receptors (e.g., GPCRs) that are expressed using the cells (e.g., CHEM-1, CHEM-2 and CHEM-3) and vector (e.g., pHS vector) of the invention are targeted and expressed on the cell surface, adopt the correct conformation and G protein coupling status, and therefore yield the correct pharmacology. To demonstrate that cell-surface receptors (e.g., GPCRs) expressed using the expression system described herein adopt the correct conformation and display the correct pharmacology in membrane binding assays, GPCR-containing membranes from CHO cells and CHEM-1 cells were isolated and subjected to radioligand competition binding assays. Specifically, membranes from CHO cells transfected with CXCR2 encoded by the pcDNA3 vector and membranes from CHEM-1 cells transfected with CXCR2 encoded by the pHS vector were assayed for ligand binding. As depicted in FIGS. 7A and 7B, CXCR2-containing membranes from CHEM-1 cells transfected with CXCR2 encoded by the pHS vector exhibited an increased Bmax in saturation binding (receptor expression level) (FIG. 7), as compared to CXCR2-containing membranes from CHO cells transfected with CXCR2 encoded by the pcDNA3 vector (compare FIGS. 7A and 7B). In addition, while CXCR2-containing membranes from CHEM-1 cells transfected with CXCR2 encoded by the pHS vector generated a two site binding curve (FIG. 8A), CXCR2-containing membranes from CHEM-1 cells transfected with CXCR2 encoded by the pHS vector generated a one site binding curve (compare FIGS. 8A and 8B). With high affinity sites for both Groα (2.2×10⁻¹⁰) and IL-8 (7.6×10⁻¹¹) observed in FIG. 8A match the high affinity sites observed in FIG. 8B, the low affinity sites (3.4×10⁻⁸ for Groα and 2.9×10⁻⁸ for IL-8) observed in FIG. 8A presumably result from the G protein uncoupling state of the GPCR trapped in ER.

The combination of high level receptor expression and subsequent functional coupling of the receptor to the endogenous promiscuous G protein in CHEM-1 cells leads to calcium mobilization without the need of co-transfecting either promiscuous or chimeric Gq proteins (FIG. 9). As shown in FIG. 9, ligand (Groα)-induced calcium mobilization through endogenous Gα₁₅ in CXCR2-transfected CHEM-1 cells exhibited a much more robust calcium flux than did ligand (Groα)-induced calcium mobilization in CXCR2-transfected CHO cells or CXCR2-transfected HL60 neutrophils (compare FIG. 9G with FIGS. 9A and 9D). In addition, unlike in CXCR2-transfected CHO cells or CXCR2-transfected HL60 neutrophils, the ligand (Groα)-induced calcium flux was not blocked by preincubation with pertussis toxin (PTX) (CXCR2 is PTX-sensitive) (compare FIGS. 9H with FIGS. 9B and 9E). However, the remaining Ca²⁺ flux can be abolished by co-transfecting with a dominant negative Gα₁₅ (DNGα₁₅) (FIG. 9I). These results indicate that the recombinant CXCR2 is not only coupling to Gα_{i/0}, but is promiscuously coupling to the endogenous Gα₁₅ in CXCR2-transfected CHEM-1 cells. The small amount of Ca²⁺ flux in FIGS. 9A and 9D are due to βγ-mediated PTX-sensitive PLCβ activation.

As described herein, co-transfection of promiscuous or chimeric Gα_{q} proteins has many associated problems, including difficulty in controlling and normalizing the amount of G protein that is transfected and constitutive activation of GPCRs leading to high background. Use of endogenous promiscuous G proteins that are highly expressed to couple the GPCR response to the calcium-signaling pathway makes the GPCR stable cell lines described herein a unique functional screening tool, irrespective of the G protein coupling status. In addition, it also provides a simple and generic method for deorphaning an orphan GPCR.

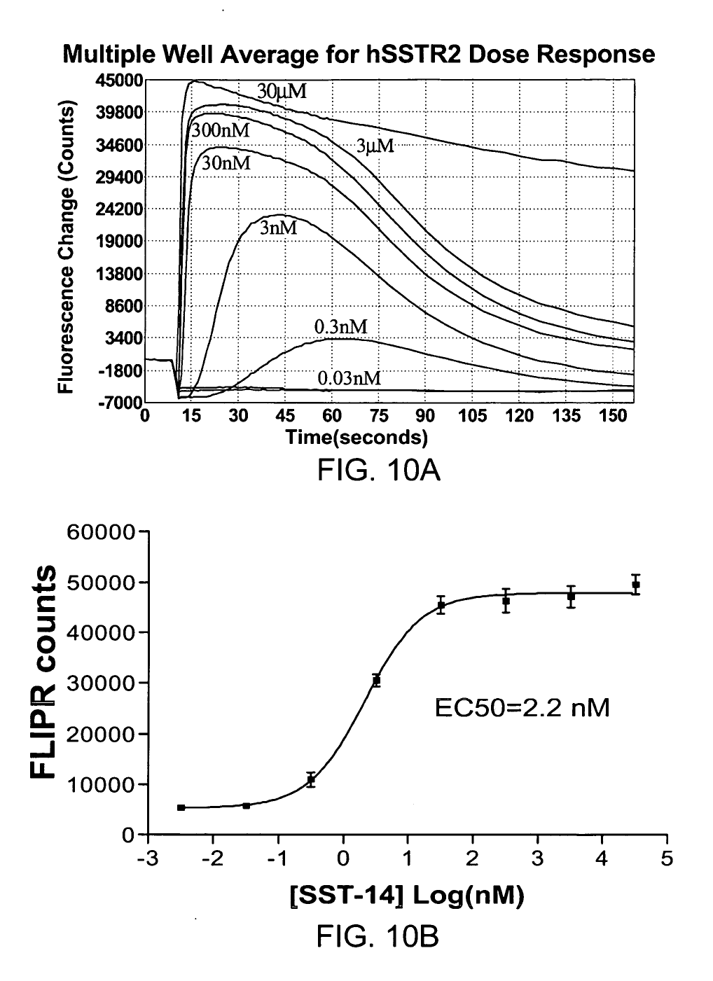

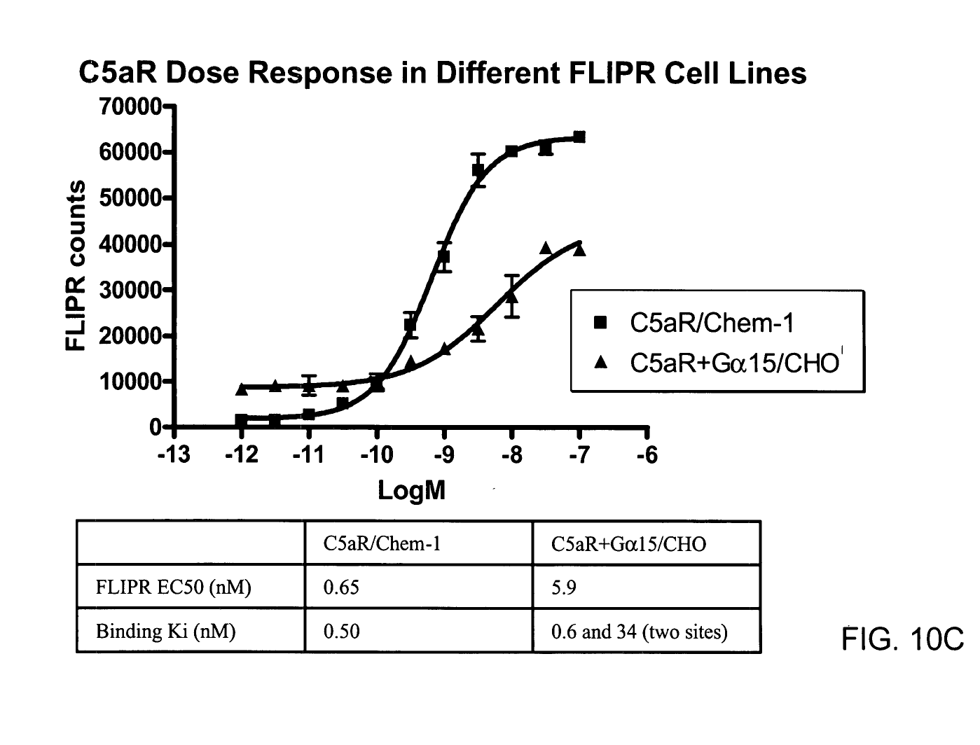

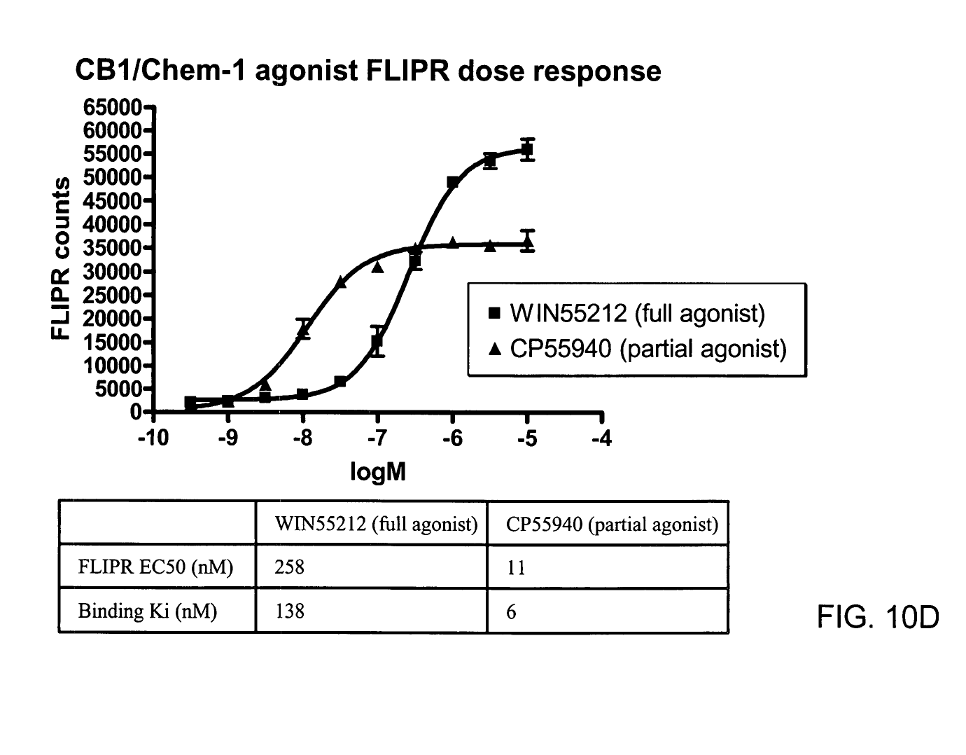

As described herein, GPCRs can be grouped into four main classes based on their G protein coupling status, termed Gα_{i/0}, Gα_{q}, Gαₛ and Gα₁₂. By redirecting the signalling pathway to PLCβ/calcium readout, the experiments described herein have validated each class of GPCRs using the dose dependent FLIPR® calcium assays. For example, for Gαᵢ-coupled receptors, it was demonstrated that it is possible to convert virtually any Gαᵢ-coupled GPCR target to the calcium pathway and still preserve its correct pharmacology and rank order of known agonists and antagonists (FIGS. 10A-10D). As depicted in FIG. 10A, which shows the FLIPR® multiple well average overlay ligand (SST-14) dose response for the Gαᵢ-coupled GPCR, SSTR2, which was transfected into CHEM-1 cells using the pHS vector, and FIG. 10B, which shows the ligand (SST-14) dose response curve, CHEM-1 cells preserve the correct pharmacology. This is evidenced by the fact that the rank order of both potency (EC50 values) and efficacy (top of the dose response curve) determined in the CHEM-1 FLIPR® assays for the indicated agonists are in agreement with published values. In addition, analysis of another GPCR, namely C5aR (a Gi-coupled receptor), and comparison of C5aR FLIPR® dose response and ligand EC50 in CHEM-1 cells and CHO cells co-transfected with C5aR and Gα₁₅, revealed that CHEM-1 cells gave an EC50 value that is consistent with the Ki binding value (FIG. 10C). In addition, FLIPR® analysis of another GPCR, the CB1 receptor, which was transfected into CHEM-1 cells, revealed a dose response curve for a full agonist (WIN55212) and partial agonists (CP55940) that had EC50 values consistent with the Ki binding value (FIG. 10D). These results demonstrate that the CHEM 1 cell line is an excellent tool for High Throughput Structure Activity Relationship (HTSAR), as it gives correct values and potency for ligands.

Another important feature of GPCR calcium-optimized cell lines (e.g., those described herein) is that the intracellular calcium mobilization is then further amplified through the endogenous store-operated Calcium Release Activated Calcium (CRAC) channel to allow maximum calcium influx and FLIPR® signal. CRAC channels themselves represent important drug targets, although they have not yet been cloned. However, many CRAC channel inhibitors have been described and are available. In addition to the depletion of internal calcium store, low nM concentration of ionomycin can also activate CRAC channels. CRAC channels are highly expressed in certain immune system cells, particularly in CHEM-2 and CHEM-3 cells, which, as described herein, also have high level of endogenous promiscuous Gα₁₆. CRAC channels are operated by intracellular calcium store through the interaction of the IP₃ receptor on the ER and the CRAC channel. CRAC channels sense the calcium concentration in the ER and open when the internal calcium store is depleted. This allows more calcium to rush into the cell and amplifies the GPCR-mediated calcium mobilization in a dose-dependent manner (FIG. 11). As depicted in FIG. 11, amplification of the intracellular Ca²⁺ signal results from activation of store-operated Calcium Release Activated Calcium (CRAC) channels that are endogenously expressed in CHEM-2 and CHEM-3 Cells.

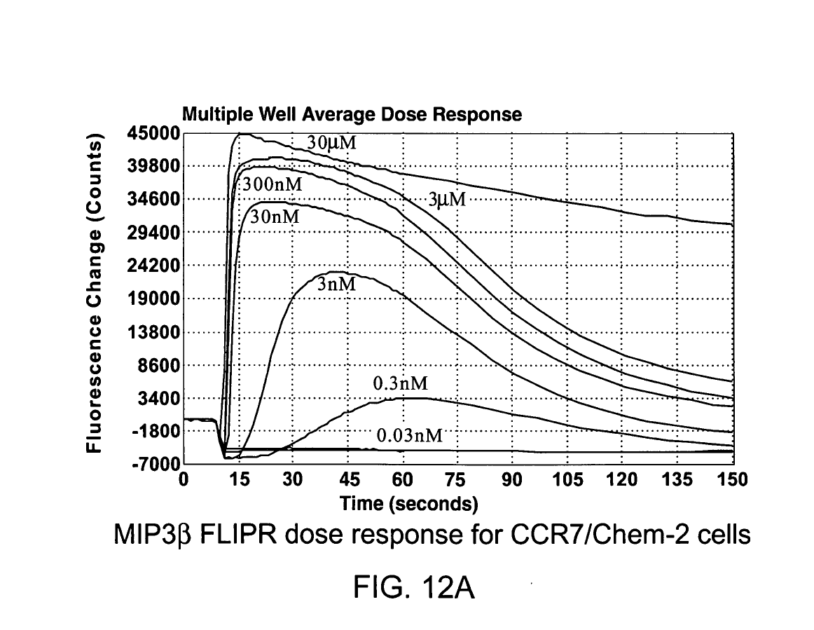

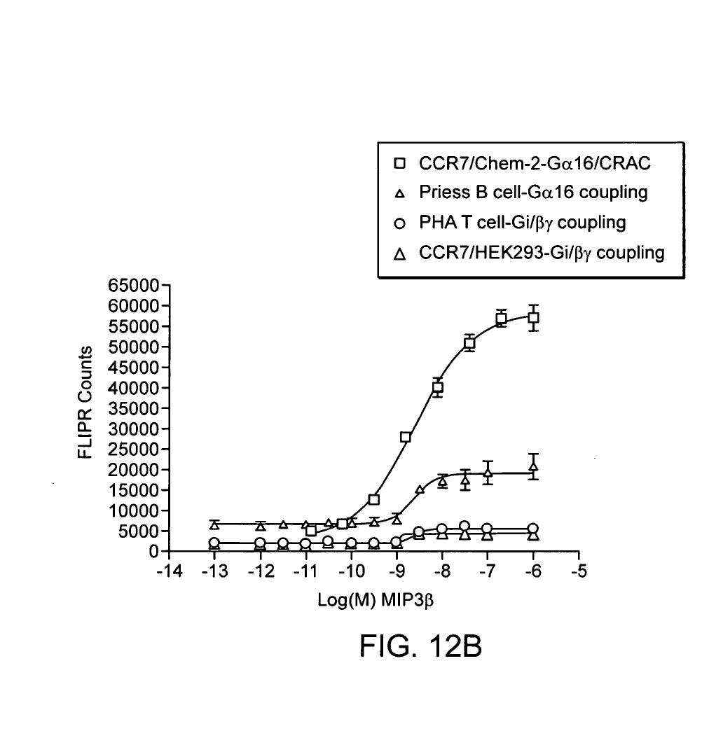

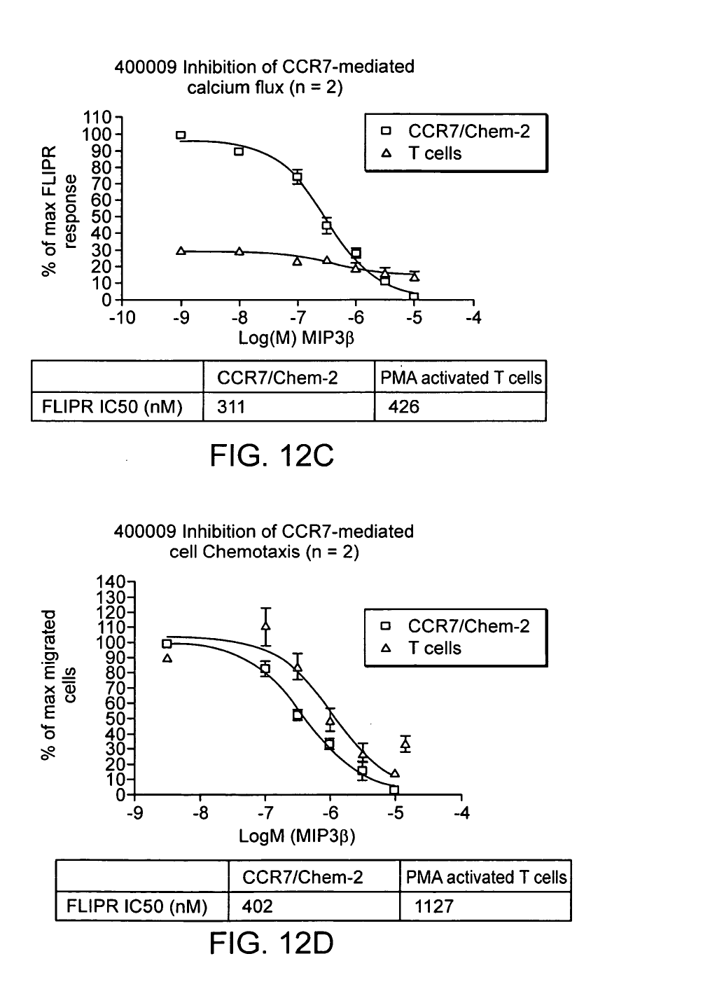

Through additional studies of FLIPR® screening using CCR7-transfected CHEM-2 stable cell lines, further validation for another Gαᵢ-coupled receptor was demonstrated. When expressed in CHEM-2 cells, CCR7 FLIPR® signal is dramatically increased through the action of endogenous Gα₁₆ and CRAC channels, while EC50 of the ligand was not changed. We have further confirmed the advance lead in the correlation between our primary FLIPR® screening assay and secondary chemotaxis assay for SAR, as the CCR7 antagonist 400009, which was identified from a FLIPR® screen using CCR7-transfected CHEM-2 cells, also inhibited CCR7-mediated chemotaxis, using both CCR7/CHEM-2 and activated T cells (FIGS. 12C and D).

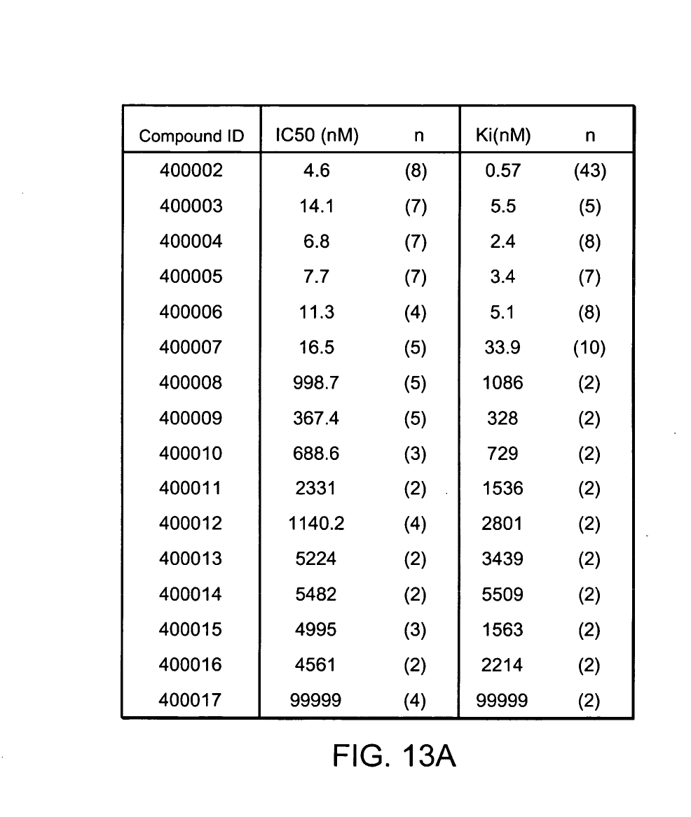

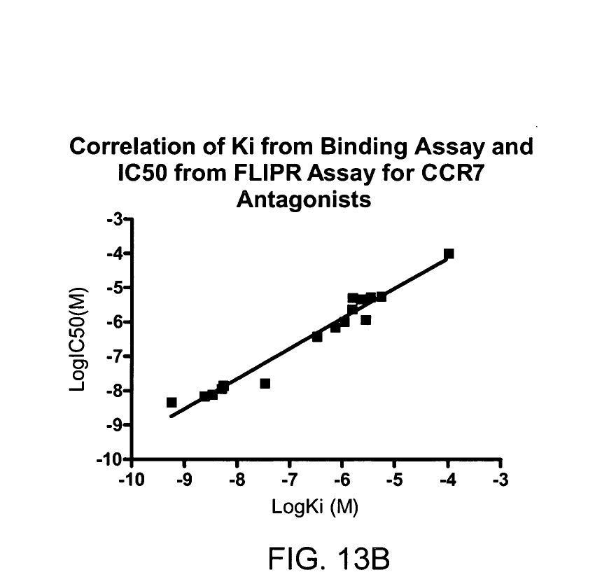

Even the correlation between the CCR7 FLIPR® assay and binding assay was determined using CCR7 small molecule antagonists that were identified from the FLIPR® screen using CCR7-transfected CHEM-2 cells (FIG. 13). The goodness of fit, r2, is 0.9616 (FIG. 13).

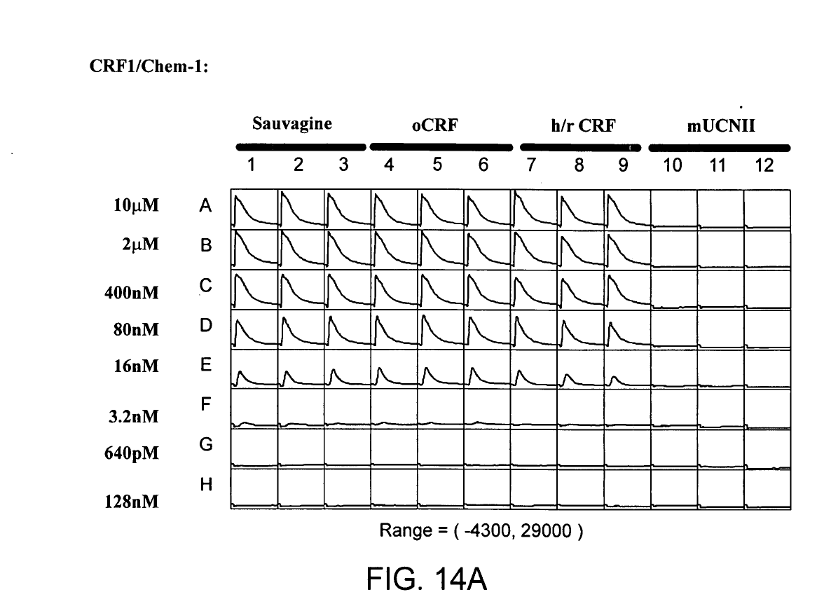

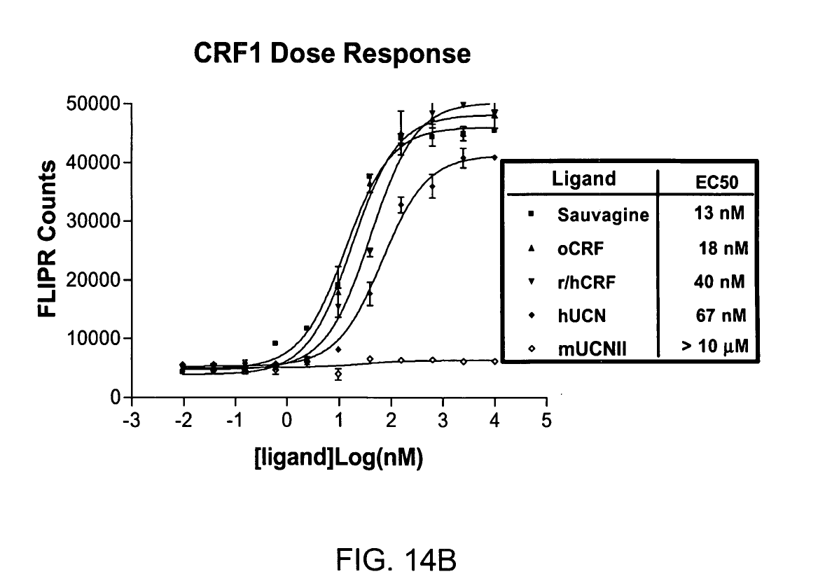

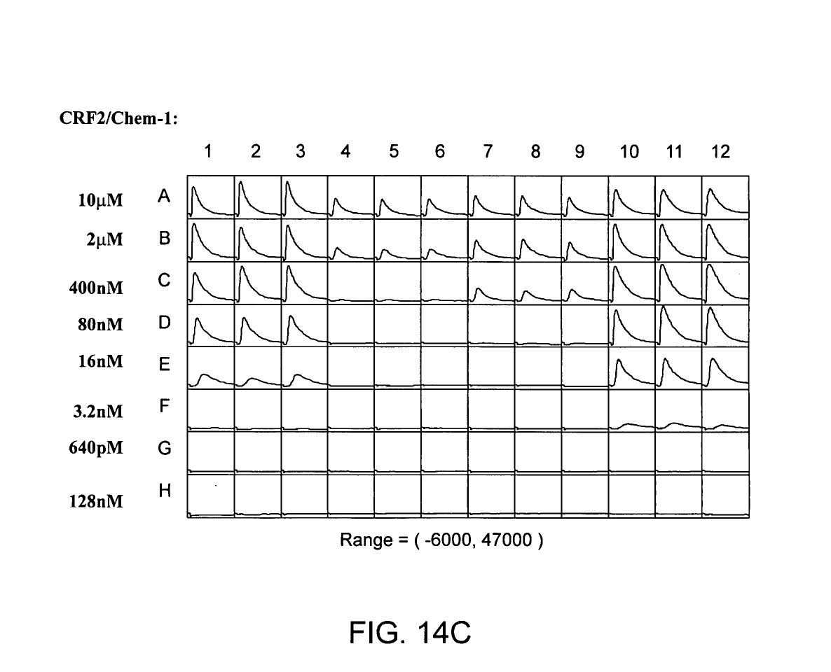

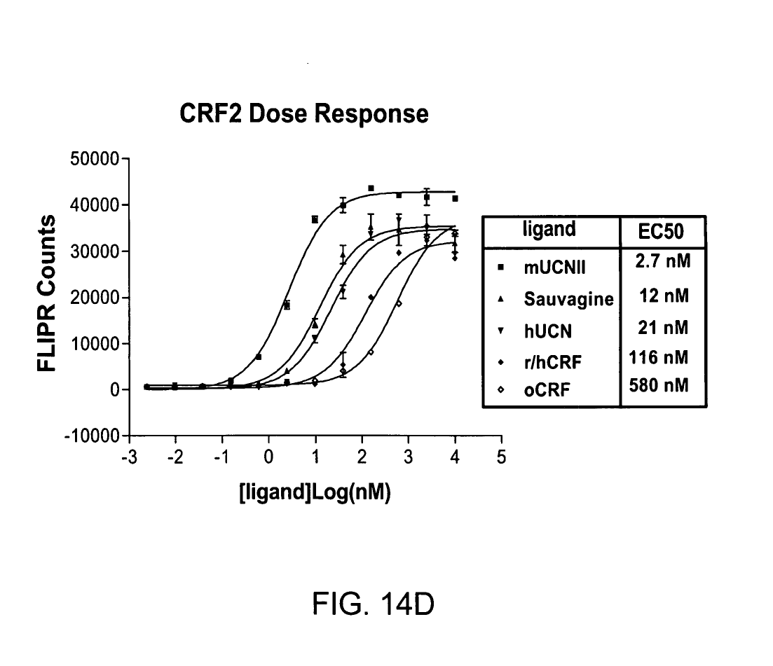

Which assay format should be selected to screen Gαₛ-coupled receptors and is it possible to adopt a single assay and platform for every GPCR target? cAMP assays are the most commonly used assays for screening Gαₛ-coupled receptors, using the endogenous pathway readout. However, such cAMP assays are not kinetic assays, they only measures the accumulative end-point cAMP that is produced, and are approximately 10-100 times more expensive than FLIPR® assays. As depicted in FIGS. 14-16, however, Gαₛ-coupled receptors coupled to Gα₁₅ or Gα₁₆ in CHEM-1 cells and could be assayed using a FLIPR® assay. FIGS. 14A and 14B demonstrate the redirection to, and validation of, FLIPR® response from Gαₛ-coupled CRF receptors in CHEM-1 cells using peptide agonists with specific rank order of potency for different receptors. Specifically, FIG. 14A depicts a FLIPR® agonist assay for CRF1 in CHEM-1 cells using peptide ligands in triplets. FIG. 14B depicts a FLIPR® agonist assay for CRF2 in CHEM-1 cells using peptide ligands (sauvagine, oCRF, h/r CRF, hUCN, mUCNII) in triplets. Peptide ligands for CRF receptors (CRF1 and CRF2) remain the same rank order of potency in FLIPR® assay using CHEM-1 cells as they were in cAMP assays, thereby indicating that CHEM-1 cell FLIPR® assays are suitable for Gαₛ-coupled receptors (FIGS. 14A and 14B).

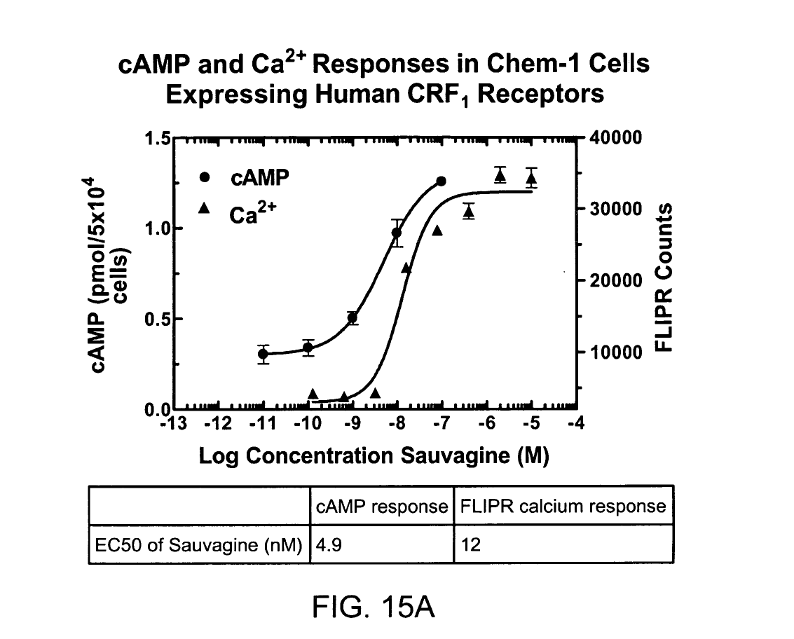

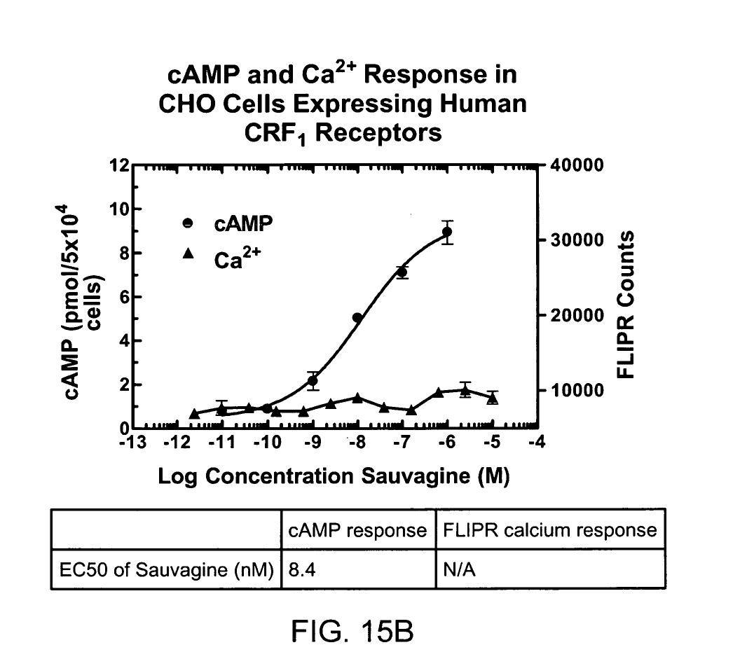

FIG. 15A is a graph depicting cAMP and Ca²⁺ responses in CHEM-1 cells expressing human CRF1 receptors. Also depicted are EC50 values of sauvagine for the cAMP response and the FLIPR® calcium response. FIG. 15B is a graph depicting cAMP and Ca²⁺ responses in CHO cells expressing human CRF1 receptors. Also depicted is the EC50 value of sauvagine for the cAMP response and the FLIPR® calcium response. In addition to the higher throughput and lower cost, another advantage of using FLIPR® assay for Gαₛ-coupled receptor is the real time kinetic measurement of the GPCR activation, compared to the cAMP assays, which measures the end-point accumulative cAMP that is produced. In FIGS. 15A and 15B, both cAMP and FLIPR® Ca²⁺ responses for CRF1 in different cell lines (CHEM-1 cells and CHO cells) were compared, and the rank order of potency remains the same in FLIPR® assay and cAMP assay. As depicted in FIG. 15A, in the CHEM-1 expression system, where most of the GPCRs are forced to couple to the promiscuous Gα₁₅, there is a much greater FLIPR® response than cAMP response, with an EC50 of 12 nM for the FLIPR® Ca²⁺ response and an EC50 or 5 nM for the cAMP response. This is consistent with the cAMP assay using CHO cells stably expressing the same receptor, in which there is no Ca²⁺ flux in the CHO cells due to the lack of promiscuous G protein coupling.

From pharmacology and SAR point of view, one of the most important advantages of using a CHEM cell FLIPR® assay for Gas-coupled receptors is the opportunity to find new series of allosteric regulators, which may be missed through conventional SAR methods. A GPCR is considered a fluidic structure; it may adopt any conformation depending upon the G protein that it is coupled to and the agonist/antagonist that is bound. Thus, forcing the CRF1 receptor to pre-couple to Gα₁₅ may force the receptor to adopt alternative conformations, which could open new binding pockets for new agents (e.g., small molecules), which could be missed using s conventional cAMP assay for the Gαₛ coupling state.

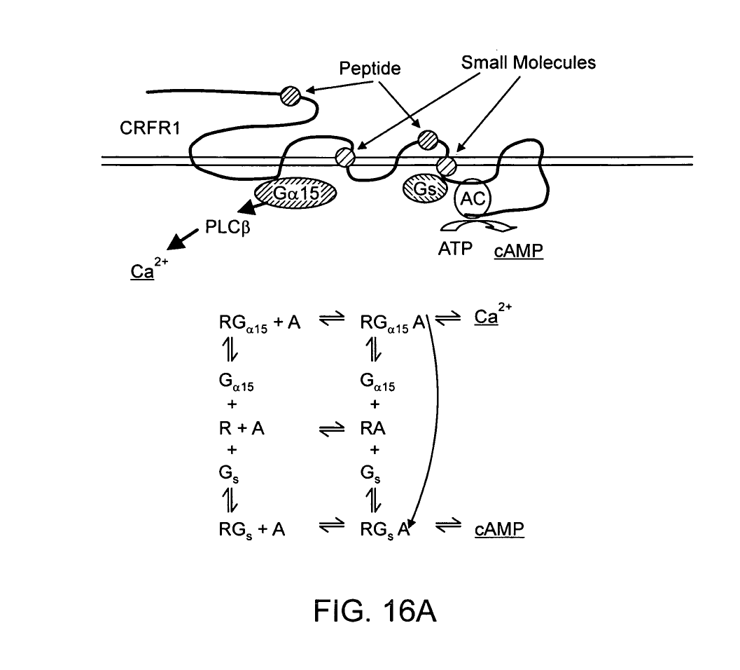

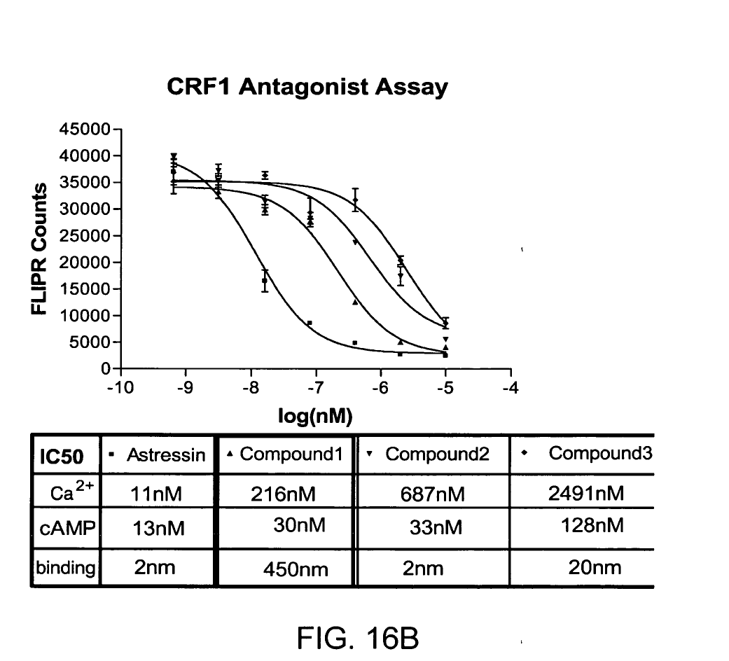

FIG. 16A is a schematic depicting a CRF1 FLIPR® antagonist assay in CHEM-1 cells. The small molecule compound was an allosteric modulator of CRF1, and was identified through FLIPR® HTSAR using CHEM-1 cells transfected with CRF1. Although this allosteric modulator has low binding affinity, it is a potent FLIPR® antagonist and still remains potent in cAMP assay and ACTH release assay. FIG. 16A is a schematic depicting the fluidic structure of GPCR theory and the conventional rigid structures of active (RA) and inactive state (R) theory. FIG. 16B depicts the results of FLIPR® antagonist assays performed with either a peptide antagonist (Astressin) or small molecule antagonists (Compound 1, Compound 2 or Compound 3) added first followed by the addition of 10 nM of the CRF1 ligand, sauvagine (˜EC50 value). Dose response inhibition of ligand-induced calcium mobilization is plotted against the antagonist concentration in Log M.

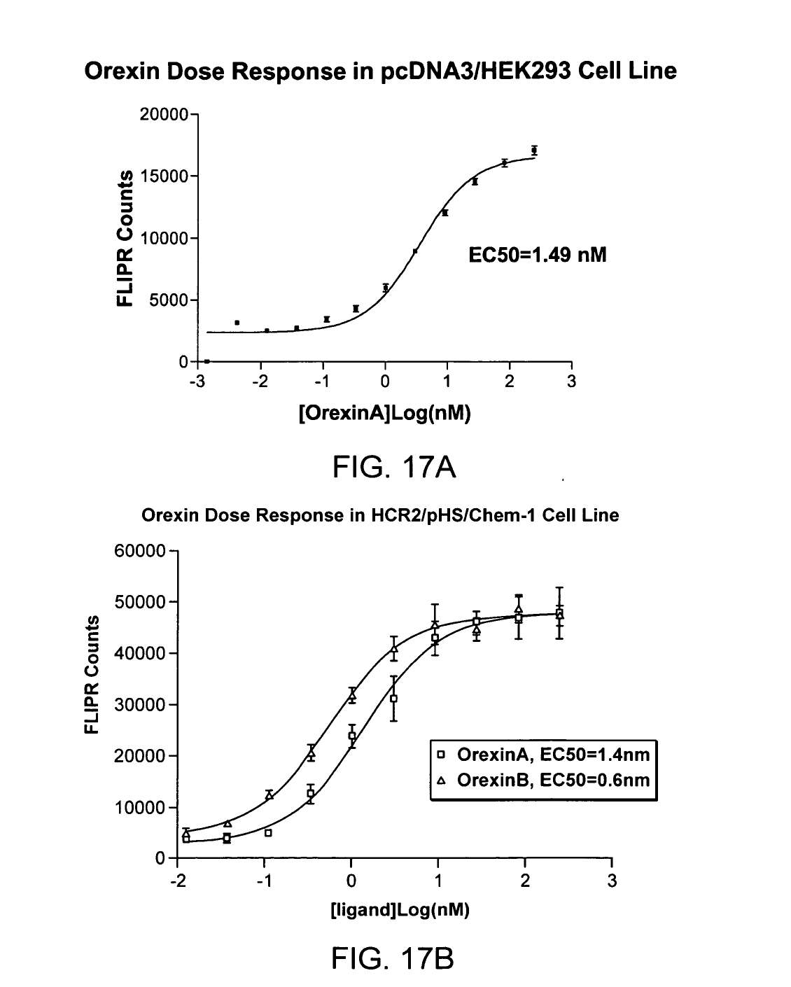

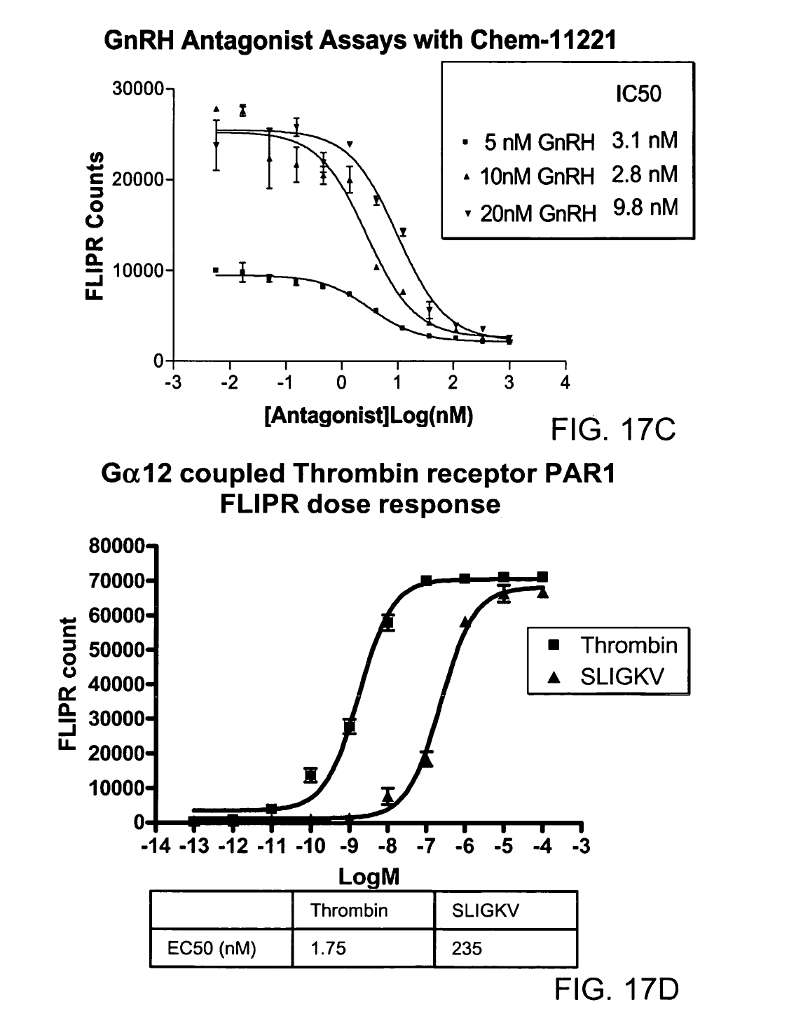

The CHEM-1 cell based FLIPR® assays were validated also for Gα_{q}- and Gα₁₂-coupled receptors. With the help of endogenous promiscuous Gα₁₅ and/or Gα₁₆ in the CHEM cells, the FLIPR® response of Gα_{q}-coupled receptors is much greater than in CHO cells, and the ligand dose response and EC50 remains the same (FIG. 17). As depicted in FIG. 17A and 17B, the CHEM-1 cell line increased the FLIPR® signal without changing EC50 of the ligands through endogenous Gα₁₅ in addition to Gα_{q}. As depicted in FIG. 17C, the Gα_{q}-coupled GNRH receptor FLIPR® antagonist assay showed the correct SAR for a small molecule antagonist (Chem-11221). As depicted in FIG., 17D, the Gα₁₂-coupled thrombin receptor PAR1 also worked and showed the correct pharmacology in the CHEM-1 cell FLIPR® assay. These results, which include FLIPR® agonist and antagonist assays for either a Gα_{q}-coupled receptor (GnRM) or Gα₂-coupled receptor (PAR1) using CHEM-1 cells, further demonstrate and validate the ability of the cells described herein (e.g., CHEM-1, CHEM-2, CHEM-3) to allow redirection of a variety of GPCR pathways (not only for Gα_{i/0}-coupled receptors and Gαₛ-coupled receptors, but also Gα_{q}-coupled receptors and Gα₁₂-coupled receptors) to a simple readout for FLIPR assays without changing the EC50 (IC50) and SAR.

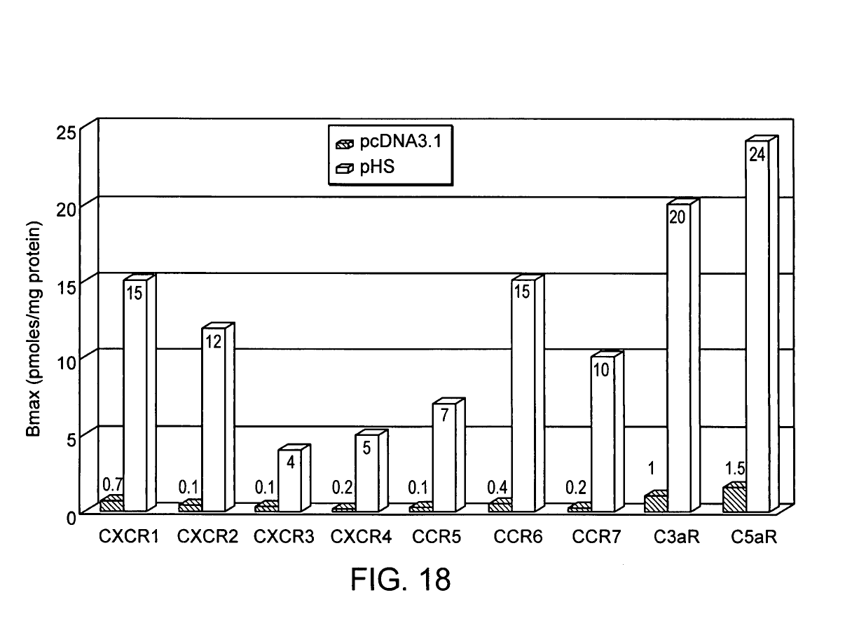

As depicted in FIG. 18, activation of a wide variety of expressed GPCRs by appropriate ligands results in strong calcium mobilization, depicted as Bmax values.

In summary, we have demonstrated a novel method of coupling GPCRs of various types (e.g., Gα_{i/0}-coupled GPCRs, Gα_{q}-coupled GPCRs, Gαₛ-coupled GPCRs and Gα₁₂-coupled GPCRs) to the PLCβ/calcium pathway using the naturally-occurring cells of the invention (e.g., CHEM-1, CHEM-2 and CHEM-3), which express high levels of endogenous promiscuous Gα₁₅ or Gα₁₆ and CRAC channels, and a novel mammalian expression vector pHS. This method is validated not only for Gα_{q}-coupled GPCRs, but also for Gα_{i/0}-coupled GPCRs, Gαₛ-coupled GPCRs and Gα₁₂-coupled GPCRs.

The relevant teachings of all publications cited herein not previously incorporated by reference, are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A cell comprising one or more endogenous promiscuous G-proteins and an exogenous nucleic acid sequence that encodes a G-protein coupled receptor (GPCR), wherein said one or more G-proteins and said GPCR are expressed at high levels.

2. The cell of claim 1, wherein said high level of GPCR expression is greater than about 100,000 receptors/cell.

3. The cell of claim 1, wherein said high level of GPCR expression is a Bmax greater than about 1 pmole/mg of protein.

4. The cell of claim 1, wherein said high level of GPCR expression is greater than about a 10-fold increase in expression of said GPCR as compared to the corresponding untransfected cell.

5. The cell of claim 1, wherein said high level of G protein expression is greater than about a 10-fold increase in expression of said G protein as compared to a control cell.

6. The cell of claim 5, wherein said control cell is a Chinese Hamster Ovary (CHO) cell.

7. The cell of claim 1 that further comprises an endogenous calcium release activated calcium (CRAC) channel.

8. The cell of claim 7, wherein said cell takes up extracellular calcium through said CRAC channel.

9. The cell of claim 1, wherein said one or more endogenous promiscuous G-proteins comprises an α0 subunit selected from the group consisting of Gα₁₅, Gα₁₆ and a combination thereof.

10. The cell of claim 1, wherein said GPCR binds to a G protein of the Gα_{q} family.

11. The cell of claim 1, wherein said GPCR binds to a G protein selected from the group consisting of a G protein of the Gα_{i/0} family, a G protein of the Gαₛ family, a G protein of the Gα₁₂ family and a combination thereof.

12. The cell of claim 1, wherein said cell exhibits an increase in intracellular free calcium in response to binding of a ligand to said GPCR.

13. The cell of claim 1, wherein said cell is a mammalian cell.

14. The cell of claim 1, wherein said cell is selected from the group consisting of:
i) a CHEM-1 (RBL-2H3) cell, deposited at the American Type Culture Collection (ATCC) as Accession Number CRL-2256;
ii) a CHEM-2 (U937) cell, deposited at the American Type Culture Collection (ATCC) as Accession Number CRL-1593.2; and
iii) a CHEM-3 (BA/F3) cell, deposited at the Deutsche Sammlung von Mikroorganismen und Zellkuturen GmbH (DSMZ) as Accession Number ACC300.

15. The cell of claim 1 further comprising a calcium-sensitive molecule.

16. The cell of claim 15, wherein said calcium-sensitive molecule is selected from the group consisting of Fluo-3, Fluo-4, Indo-1, Fura-2, Rhod-2, Oregon green and calcium green-2.

17. The cell of claim 15, wherein said calcium-sensitive molecule is a calcium-sensitive detectable protein.

18. The cell of claim 17, wherein said calcium-sensitive detectable protein is a bioluminescent protein.

19. The cell of claim 18, wherein said bioluminescent protein is selected from the group consisting of luciferase, aequorin, apo-aequorin and a derivative or mutant of any of the foregoing.

20. The cell of claim 17, wherein said calcium-sensitive detectable protein is encoded by an exogenously-introduced nucleic acid.

21. The cell of claim 1, wherein said nucleic acid sequence encoding a G-protein coupled receptor (GPCR) is present in a plasmid.

22. The cell of claim 21, wherein said plasmid is pHS vector, deposited at the American Type Culture Collection (ATCC) as Accession Number PTA-6986.

23. The cell of claim 1, wherein said nucleic acid sequence comprises a non-CMV promoter that is operably linked to said GPCR.

24. The cell of claim 1, wherein said nucleic acid sequence further comprises an endoplasmic reticulum (ER) export signal.

25. The cell of claim 1, wherein said cell is an adherent cell.

26. The cell of claim 1, wherein said cell is a non-adherent cell.

27. The cell of claim 1, wherein said cell endogenously expresses less than 1000 GPCR/cell.

28. The cell of claim 20, wherein said exogenously-introduced nucleic acid encoding a calcium-sensitive detectable protein and said nucleic acid sequence encoding a GPCR are present in a plasmid.

29. pHS vector, deposited at the American Type Culture Collection (ATCC) as Accession Number PTA-6986.

30. A cell transfected with pHS vector, deposited at the American Type Culture Collection (ATCC) as Accession Number PTA-6986.

31. A method of identifying an agent that increases activity of a G-protein coupled receptor (GPCR) comprising:
1) combining:
a) a cell that comprises one or more endogenous promiscuous G-proteins and an exogenous nucleic acid sequence that encodes a G-protein coupled receptor (GPCR), wherein said one or more G-proteins and said GPCR are expressed at high levels; and
b) a test agent; and
2) detecting activity of said GPCR,
wherein an increase in activity of said GPCR, relative to a control, indicates that said test agent increases activity of said GPCR.

32. The method of claim 31, wherein said agent is an agonist.

33. The method of claim 31, wherein said increase in activity of said GPCR is an increase in intracellular free calcium.

34. The method of claim 33, wherein said increase in activity of said GPCR is detected using a Fluorometric Imaging Plate Reader (FLIPR®) or aequorin technology.

35. A method of identifying a ligand of a G-protein coupled receptor (GPCR) comprising:
1) combining:
a) a cell that comprises one or more endogenous promiscuous G-proteins and an exogenous nucleic acid sequence that encodes a G-protein coupled receptor (GPCR), wherein said one or more G-proteins and said GPCR are expressed at high levels; and
b) a test ligand; and
2) detecting activity of said GPCR,
wherein an increase in activity of said GPCR, relative to a control, indicates that said test ligand is a ligand of said GPCR.

36. The method of claim 35, wherein said GPCR is an orphan GPCR.

37. A method of identifying an agent that modulates activity of a G-protein coupled receptor (GPCR) comprising:
1) combining:
a) a cell that comprises one or more endogenous promiscuous G-proteins and an exogenous nucleic acid sequence that encodes a G-protein coupled receptor (GPCR), wherein said one or more G-proteins and said GPCR are expressed at high levels;
b) an agent that activates said GPCR; and
c) a test agent; and
2) detecting activity of said GPCR,
wherein an alteration in activity of said GPCR, relative to a control, indicates that said test agent modulates activity of said GPCR.

38. The method of claim 37, wherein said test agent decreases activity of said GPCR.

39. The method of claim 37, wherein said test agent increases activity of said GPCR.

40. The method of claim 37, wherein said modulation of activity of said GPCR is an alteration in intracellular free calcium.

41. The method of claim 40, wherein said modulation of activity of said GPCR is detected using a Fluorometric Imaging Plate Reader (FLIPR®) or aequorin technology.

42. A method of expressing a G-protein coupled receptor (GPCR) in a cell, comprising transfecting said cell with a nucleic acid sequence encoding said GPCR, wherein said cell comprises one or more endogenous promiscuous G-proteins, and wherein said one or more G-proteins and said GPCR are expressed at high levels.

43. The method of claim 41, wherein said nucleic acid sequence encoding said GPCR is present in the pHS vector, deposited at the American Type Culture Collection (ATCC) as Accession Number PTA-6986.

44. A method of measuring an alteration in intracellular calcium in a cell comprising
1) combining:
a) a cell that comprises one or more endogenous promiscuous G-proteins and an exogenous nucleic acid sequence that encodes a G-protein coupled receptor (GPCR), wherein said one or more G-proteins and said GPCR are expressed at high levels; and
b) an agent that activates said GPCR; and
2) measuring intracellular calcium in said cell.

45. A method of coupling a G-protein coupled receptor (GPCR) to the PLCβ pathway comprising
1) combining:
a) a cell that comprises one or more endogenous promiscuous G-proteins and an exogenous nucleic acid sequence that encodes a G-protein coupled receptor (GPCR), wherein:
i) said one or more G-proteins and said GPCR are expressed at high levels; and
ii) said one or more G-proteins are selected from the group consisting of a G protein of the Gα_{i/0} family, a G protein of the Gαₛ family and a G protein of the Gα₁₂ family; and
b) an agent that activates said GPCR,
under conditions in which said GPCR is activated.


SEQUENCE LISTING (5 sequences)

SEQ ID NO: 1 - DNA, 17 bp, Artificial Sequence
  oligonucleotide primer
atggcccggtccctgac

SEQ ID NO: 2 - DNA, 18 bp, Artificial Sequence
  oligonucleotide primer
gtcacagcaggttgatct

SEQ ID NO: 3 - protein, 10 aa, Artificial Sequence
  ER export signal
FEYEFNEVEF

SEQ ID NO: 4 - DNA, 758 bp, Artificial Sequence
  Spleen Focus Forming Virus (SFFV) LTR
tgcagacacaacactgctctacgggcatgagtggccaccttccattttttttttaagtgtgtgtgctgacgtcacaagaactcaggtactcttacttcctacatggtacgagttcttaccacttagccatttcttcatcctgaaagaccccaccaagttgcttagcctgatagccgcagtaacgccattttgcaaggcatggaaaaataccaaaccaagaatagggaagttcagatcaagggcgggtacacgaaaacagctaacgttgggccaaacaagatatctgcggtaagcagtttcggccccggcccggggccaagaacagatggtccccagatatggcccaaccctcagcagtttcttaagacccatcagatgtttccaggctcccccaaggacctgaaatgaccctgtgccttatttgaattaaccaatcagcccgcttctcgcttctgttcgcgcgcttttgcttcccgagctctataaaagagctcacaacccctcactcggcgcgccagtcctccgacagactgagtcgcccgggtacccgtgttcccaataaagcctcttgctgattgcatccgaatcgtggactcgctgatccttgggagggtctcctcagattgattgactgcccacctcgggggtctttcatttgggggctcgtccgggatttggagacccccgcccagggaccaccgacccaccgtcgggaggtaagctggccagcgatcgttttgtctccgtctctgtctttg

SEQ ID NO: 5 - DNA, 836 bp, Artificial Sequence
  CXCR2-GFP Fusion Protein
atggccaascagatcctgaagaacacctgcctgcaggaagtgatgagctacaaggtcaacctggagggcatcgtgaacaaccacgtctttaccatggagggctgcggcaagggcaacatcctgttcggcaaccaattggtgcagatccgcgtgaccaagggcgcccccctgcccttcgccttcgacatcgtgagccccgccttccagtacggcaaccgtacgttcacaaagtaccccaacgacatcagcgactacttcatccagagcttccccgccggcttcatgtacgagcgcaccctgcgctacgaggacggcggcctggtggagatccgcagcgacatcaacctgatcgaggacaagttcgtgtaccgcgtggagtacaagggcagcaacttccccgacgacgggcccgtgatgcagaagaccatcctgggcatcgagcccagcttcgaggccatgtacatgaacaacggcgtgctggtgggcgaggtcatcctggtgtacaagctgaacagcggcaagtactacagctgccacatgaagaccctgatgaagagcaagggcgtggtcaaggagttccccagctaccacttcatccagcaccgcctggagaagacctacgtggaggacggcggcttcgtggagcagcacgagaccgccatcgcccagatgaccagcatcggcaagcccctgggatctctgcacgagtgggtgtaracccgctgatcagcctcgactgtgccttctagttgccagccatctgttgtttgcccctcccccgtgccttccttgaccctggaaggtgccactcccactgtcctttcctaataaaatga